(19) United States
(12) Patent Application Publication
(10) Pub. No.: US 2008/0305049 A1
(43) Pub. Date: Dec. 11, 2008

(54) Mri Contrast Agents for Diagnosis and Prognosis of Tumors

(76) Inventors: Hadassa Degani, Rehovot (IL); David Stein, Rehovot (IL)

Correspondence Address: BROWDY AND NEIMARK, P.L.L.C.;624 NINTH STREET, NW, SUITE 300, WASHINGTON, DC, 20001-5303, US

(21) Appl. No.: 11/815,072

(22) PCT Filed: Jan. 31, 2006
(86) PCT No.: PCT/IL06/00124

§ 371 Date: May 9, 2008

Related U.S. Application Data
(60) Provisional application No. 60/647,821, filed on Jan. 31, 2005.

(51) Int. Cl. A61K 49/10; C07J 43/00; C07F 19/00; C07D 213/72; C07D 471/04

(52) U.S. Cl. 424/9.363; 540/109; 540/3; 546/308; 546/12; 540/472; 424/9.361

(57) ABSTRACT

The invention relates to bifunctional conjugates comprising a receptor ligand moiety and a metal binding moiety and complexes thereof with paramagnetic lanthanide or transition metals, and to the use of the metal complexes as contrast agents in magnetic resonance imaging (MRI) of tumors and other abnormalities.

### FIELD OF THE INVENTION

The present invention relates to bifunctional conjugates comprising a receptor ligand moiety and a metal binding moiety and complexes thereof with paramagnetic lanthanide or transition metals, and to the use of the metal complexes as contrast agents in magnetic resonance imaging (MRI) of tumors and other abnormalities.

### BACKGROUND OF THE INVENTION

Targeted cellular delivery of molecules to specific tissues is an important goal in pharmacology and medicinal chemistry. Achieving this requires harnessing and applying molecular level recognition events prevalent in the desired tissue type. Cancers such as breast and prostate cancer, most frequently express the steroid receptors of their origin and can accumulate molecules that have high binding affinities for the receptors, namely, receptor ligands. Therefore, these ligands that contain a second functional group that may be used for diagnostic imaging are exciting targets in the field of molecular imaging.

### Estrogen and Breast Cancer

The growth and progression of malignant transformation may depend on the presence and availability of specific hormones. For example, the growth of a large fraction of breast cancers is stimulated in the presence of estrogen (Leclercq et al., 2002; Osborne et al., 1996; MacGregor and Jordan, 1998). The estrogen receptors act also as potent transcription factors for a variety of genes, some of which stimulate tumor growth (Leclercq et al., 2002; MacGregor and Jordan, 1998; Muramatsu and Inoue, 2000). Hormonal therapy designed to reduce serum estrogen levels or to block the effects of estrogens on the cancer cells by means of selective estrogen modulators (SERMs), are used clinically to improve survival (Osborne et al, 1996; MacGregor and Jordan, 1998). Furthermore, hormonal therapy of breast cancer with tamoxifen has been shown to be effective in preventing this malignancy (Fisher et al, 1998).

Estrogen receptor (ER) is a well-established marker of breast cancer hormone sensitivity (MacGregor and Jordan, 1998; McGuire, 1978). However, only about two thirds of these patients respond to antiestrogen therapy, while about 10% of ER negative patients also respond to this therapy. The reason for such failure for ER positive tumors as well as the response of ER negative tumors is still unknown (Leclercq et al., 2002). Part of it could be associated with variation in the ability to accurately measure the level of the receptors throughout the whole tumor.

Until recently, it was considered that all biological actions of estrogens and antiestrogens were manifested through a single estrogen receptor subtype, Erα. However, the discovery of a second ER subtype, ERβ, significantly increased the biological complexity of estrogen action (Kuiper et al., 1996). The ERβ protein is distributed in various estrogen target tissues and is detected in both normal and malignant breast cells (Fuqua et al., 1999). In the mammary gland, ERβ levels are high and remain unchanged from birth to adulthood, throughout pregnancy, lactation and involution. ERα levels, however, vary greatly from one stage to another, possibly reflecting their regulation by circulating estrogen levels (Saji et al., 2000). Studies using ERβ null mutant mice have shown that ERβ is dispensable for mammary growth and differentiation (Shyamala et al., 2002). However, during carcinogenesis the ratios of ERα and ERβ gene expression change, in accord with the finding that ERβ mRNA expression is much lower than ERα in most breast tumors (Saji et al., 2000; Leygue et al., 1998).

The expression of ERα varies tremendously with cell type, cell cycle stage as well as cell's sparsity and confluency. The amount of available ERα in the cell is controlled by a balance between synthesis and degradation. ER stability is also influenced by the nature of the bound ligand. There is, therefore, a strong scientific relevance to develop a non-invasive, standardized imaging method that would quantify ERα level in vivo and relate it to functional changes, such as enhanced metabolism, growth and vascularization.

From the clinical aspect, estrogen receptor status can predict the response to adjuvant endocrine therapy with selective estrogen receptor modulators like tamoxifen, and together with the progesterone receptor level they predict the likelihood of recurrence and survival. This is particularly important in view of recent results showing that the overall rise in breast cancer incidence rates seems to be primarily a result of the increase in the incidence of estrogen receptor positive tumors (Li et al., 2003). Recent data also suggest that ER expression of normal breast tissue is fairly consistent over time and support the notion that over-expression of ER in normal epithelium is a constant feature of the high risk breast (Khan et al., 2002). Hence, the capacity to map ER may eventually indicate high risk for cancer development. In view of the above pivotal role of ER in prevention, management and treatment of breast cancer, an accurate, standardized and in vivo method to determine its level throughout the entire tumor would highly improve patients' care.

### Measuring ER Levels

The current clinical methods that measure ERα are based on two strategies. The first one, used for many years until recently, involves the competitive binding of radiolabeled ligand; the second one, which is frequently used today, relies on recognition of the receptor by immunohistochemical methods (Harvey et al., 1999). In addition to experimental problems resulting from uneven as well as non-specific staining, the analysis is subjective and semi-quantitative (Barnes et al., 1998). Furthermore, these in vitro assays are conducted on a biopsy sample of the primary tumor, whereby the receptor distribution is often heterogeneous. Defects in specimen preservation that lead to protein degradation may also distort the final results (Katzenellenbogen et al., 1995). Another drawback is the lack of standardization among different laboratories.

Several studies have been previously performed to investigate the possibility of in vivo imaging of ER by positron emission tomography (PET), single photon emission computed tomography (SPECT) and by conventional nuclear medicine (Katzenellenbogen et al., 1995). PET, with fluorine-18 labeled 16-fluoroestradiol, has been shown to image primary and metastatic breast cancer (Mortimer et al., 1996). Other clinical studies have used planar scintography and SPECT with I¹²³-labeled ER ligands (Rijks et al., 1997). Studies of technetium-99ₘ tamoxifen conjugates have also been reported (Hunter and Luyt, 2000). So far, however, none of these nuclear imaging studies have been very successful clinically because of the low target to non-target tissue image contrast. Although MRI was not applied thus far to detect ER, molecular imaging of tissue targets using MRI contrast agents tagged to antibodies has already been previously employed (Curtet et al., 1998; Vera et al., 1995).

Small Molecules that Bind ER Specifically

A set of compounds that can specifically interact with the estrogen receptor and serve as diagnostic imaging agent for estrogen receptor positive (ER⁺) tumors, are estrogen- or antiestrogen-derived metal complexes. Jackson et al. (2001) prepared a range of metallo-estrogens based on 17α-ethynylestradiol. The metal binding domains in these estrogens-derived steroid metal complexes consisted of a pyridyl moiety linked to the ethynyl radical at position 4 and substituted at positions 2 and 6 by methylthio, benzylthio or carboxyl groups. These compounds exhibited effective binding to ER and were delivered across the cell membrane into MCF-7 cells (Jackson et al., 2001). In the whole cell assays, despite their charge, the Pd and Pt metal complexes exhibited similar or even enhanced receptor binding affinities as compared to their corresponding neutral free ligands. A key feature is that a single estrogen conjugate may be used to bind a range of metals.

U.S. Pat. No. 6,080,839 discloses a labeling reagent suitable for labeling of biospecific binding reactant using solid phase synthesis, said labeling reactant comprises a lanthanide metal-binding moiety, 2,6-bis[N,N-bis-(tert-butoxycarbonylmethyl)aminomethyl]pyridyl, linked through a bridging group to an Fmoc protected amino acid. Also disclosed is an estradiol labeled with four Europium(III)-complexed labeling reactants, bound to estradiol at position 6. The labeling reactant is said to be applicable for fluorescent labeling, however, no specific biological application is disclosed.

### SUMMARY OF THE INVENTION

The present invention relates to a bifunctional conjugate of the general formula I, II or III hereinafter, and metalated complexes thereof with a paramagnetic lanthanide or transition metal.

The metalated conjugates of the invention are particularly useful as magnetic resonance imaging (MRI) contrast agents that bind specifically and with high affinity to receptors associated with malignant tumors and other abnormalities and thus enable MR imaging of said receptors both in vitro and in vivo.

In a preferred embodiment, the MRI contrast agent of the invention comprises an estrogen receptor (ER) specific ligand such as 17β-estradiol or tamoxifen, which is useful for the diagnosis of breast cancer, for prognosticating the effectiveness of hormonal therapy and chemotherapy, and for the follow up of such therapies in breast cancer.

The present invention further relates to molecular MRI methods for diagnosis of a tumor, for prognosis or follow up of treatment of a tumor by a chemotherapeutic or hormonal agent, and for monitoring a chemotherapeutic drug or an anti-hormonal agent delivery to a malignant tumor.

### BRIEF DESCRIPTION OF THE FIGURES

FIG. 1 shows the chemical structure and ¹H NMR spectrum of the ER-ligand-17β-estradiol conjugate herein designated Compound 6.

FIGS. 2A-2C show proliferation of estrogen-receptor positive (ER⁺) T47D (FIG. 2A) and ER⁺ MCF7 (FIG. 2B), and ER⁻ MDA-MB-231 (FIG. 2C) human breast cancer cells in estrogen-free medium (control) and in media supplemented with 17β-estradiol, the unmetalated 17β-estradiol conjugate 6 or its Gd complex 7, at the indicated concentrations. The number of cells was determined spectrophotometrically using the MTT method.

FIG. 3 is a graph showing the dose effect of the Gd complex 7 (in water) on the proliferation of ER⁺ T47D human breast cancer cells. Cells were cultured for 6 days in estrogen-free and phenol red-free medium (DMEM) containing various concentrations of 7. The number of cells was determined spectrophotometrically using the MTT method.

FIG. 4 is a plot of the change in the water T₁ relaxation rate (relaxivity) in the presence of increased concentrations of the Gd complex 7 measured at 4.7 Tesla employing a spin echo sequence with a varying repetition time-TR (11 TRs, from 50 to 15,000 msec) and fixed echo time of 23 msec. R1_{c=0} represents the T₁ relaxation rate of the ligand-free water.

FIGS. 5A-5B are a T₂ weighted MR image (FIG. 5B) and a map of apparent concentration of the Gd complex 7 (FIG. 5A) in a body slice of a CD1-NU immunodeficient female mouse implanted with an orthotopic MCF7 breast tumor.

FIG. 6 shows MRI signal enhancement values (%) obtained from T₁-weighted images generated after a bolus administration of 0.4 mmol/kg of the Gd complex 7 in the orthotopic MCF7 breast tumor and muscle tissue. The images were collected at the indicated time points. The percent (%) enhancement was defined as {[I(t)−I(0)]/I(0)}×100. I(t) is signal intensity at time and I(0) is signal intensity before contrast agent administration.

FIGS. 7A-7B show a T₂-weighted image (FIG. 7A) and a time course of the T₁ relaxation rates (FIG. 7B) in the bladder, orthotopic MCF7 breast tumor and muscle of a female immunodeficient mouse after a bolus administration of a low dose of the Gd complex 7 (0.024 mmol/kg).

FIG. 8A shows time-dependent changes in T₁ relaxation rate, R₁, in orthotopic MCF7 breast tumor, muscle tissue and bladder, after bolus administration of the Gd complex 7 (0.024 mmol/kg) into the tail vein of a female immunodeficient CD1-NU mouse. FIG. 8B shows a change in apparent concentration (calculated from the measured relaxation rates) 24 hours after administration of the Gd complex 7. The T₁ values present average values over the whole tumor volume, bladder volume and region of interest (ROI) of muscle (demonstrated in FIG. 8A).

FIG. 9 shows immunohistochemical staining with a monoclonal antibody of ERα of orthotopic MCF7 breast tumor. The staining was performed using NCL-ER-6F11/2.

FIG. 10A is a Western blot depicting the down regulation of the estrogen receptor (ER)-α in MCF7 cells 6 hours after treatment with 17-β estradiol (30 nM) or the pure antiestrogen ICI-182780 (1 μM). Compounds 6 and 7 also induce ER reduction whereas tamoxifen and Compounds 15 and 16 do not affect ER level. The expression level of tubulin, which remains constant, served as a reference for quantification of the changes in ER. FIG. 10B is a graph showing the quantitative analysis of the blots in terms of ER expression relative to tubulin expression under the various treatments as indicated in the figure.

FIGS. 11A-11E show magnetic resonance images and their processing exhibiting the uterine endometrial in an overiectomized female rat before and after a bolus administration of Gd complex 7 (0.024 mmol/kg). FIGS. 11A and 11B show the T2-weighted images before and after (5 hours) the bolus administration of Gd complex 7. The corresponding 3D automatically delineated right horns are shown in FIGS. 11C and 11D. FIG. 11E shows the corresponding changes in the volume during the entire time course.

### DETAILED DESCRIPTION OF THE INVENTION

The present invention provides a bifunctional conjugate comprising a moiety of a receptor ligand or a chemotherapeutic drug moiety and a metal binding moiety, and complexes thereof with paramagnetic lanthanide and transition metals.

The novel conjugates and metal complexes of the present invention and the intermediates used in their synthesis have been assigned herein numerals from 1 to 16 and are represented throughout in the specification, in the Schemes I and II, and in the claims, by these numerals in bold. The chemical structures of said compounds I-16 are depicted in Schemes I and II found at the end of the description, just before the References. The nonmetalated conjugates 5, 6, 14, 15 and the Gd complexes 7 and 16 are novel compounds.

The conjugates of the present invention comprise a receptor ligand moiety or a chemotherapeutic drug moiety and a metal binding moiety, and are of the general formula I, II or III:

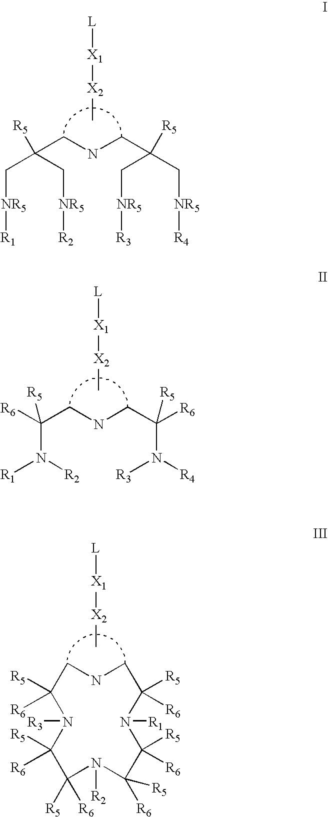

wherein

L is a moiety of a ligand of a receptor associated with malignant tumors or a chemotherapeutic drug moiety;

X₁ is a C₂-C₁₀ hydrocarbylene chain;

X₂ is phenylene or a covalent bond;

R₁ to R₄ in the conjugates of formulas I and II and R₁ to R₃ in the conjugate of formula III each is H, (C₁-C₄) alkyl or CH₂R₇;

R₅ and R₆ each is H or (C₁-C₄) alkyl;

R₇ is a radical selected from the group consisting of —COOR₈, —COO⁻,

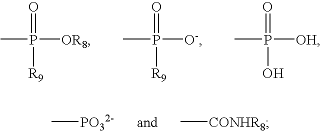

R₈ and R₉ each is H, (C₁-C₄) alkyl, phenyl or benzyl, wherein the phenyl or benzyl can be substituted by at least one radical selected from the group consisting of halogen, (C₁-C₄) alkyl and OR₅;

the dotted lines, when present, define that the N atom and the 2 adjacent C atoms are part of a monocyclic or polycyclic ring;

and complexes of a conjugate of formula I, II or III, wherein R₁ to R₄ each is —CH₂R₇ and R₇ is —COOR₈, —COO⁻, PO₂(R₈)(R₉), PO₃²⁻, PO₃H₂ or —P(R₉)O₂⁻, wherein R₈ and R₉ is as defined above, with a paramagnetic lanthanide metal selected from the group consisting of Gd (III), Eu(III), Dy(III), Tb(III), Tm(III), Yb(III) and Pr(III), and of a conjugate of formula I, II or III, wherein R₁ to R₄ each is H, (C₁-C₄) alkyl or —CH₂R₇ and R₇ is as defined above, with a paramagnetic transition metal selected from the group consisting of Mn(II), Co(III), Ni(III), Fe(II) and Fe(III).

The metal binding moiety of the conjugate of the invention is bound to the receptor ligand or chemotherapeutic drug moiety L via the linker units X₁ and X₂, and coordinates very strongly the metal in the metalated complex, i.e., with a very high association constant.

In one preferred embodiment of the invention, L is a moiety of a ligand of a steroidal hormone receptor associated with malignant tumors such as an estrogen receptor (associated with breast and ovarian cancers), the androgen receptor (associated with prostate cancer), and the progesterone receptor (associated with breast and ovarian cancers). In another embodiment of the invention, L is a moiety of a ligand of a non-steroidal hormone receptor associated with malignant tumors such as, but not limited to, luteinizing hormone (LH)/human chorionic gonadotropin (hCG) receptors (associated with ovarian tumors and testis tumors), somatostatin receptor (associated with neuroendocrine tumors), or other receptors such as a retinoic acid receptor (RARs), of which three subtypes (alpha, beta, gamma) have been identified (associated with several cancers, e.g. neuroblastoma, cervical cancer, prostate cancer).

In one preferred embodiment, the ligand L is a moiety of a ligand of a member of the steroid receptor family, i.e., the estrogen receptor (ER)-α, the androgen receptor or the progesterone receptor. In a more preferred embodiment, the steroid receptor is the estrogen receptor-α and the ER-α ligand may be a steroidal ligand such as 17β-estradiol, estrone, estriol and derivatives thereof, or a non-steroidal ligand such as the estrogen antagonist tamoxifen and tamoxifen analogs. In another embodiment, the receptor is the androgen receptor and L is a moiety of testosterone. In a further embodiment, the receptor is the progesterone receptor and L is a moiety of a progestin, preferably progesterone.

In another embodiment, L is a moiety of a polypeptide hormone ligand such as, but not limited to, luteinizing hormone, human chorionic gonadotropin, human growth hormone, or somatostatin or a somatostatin analog, e.g. octreotide.

In a further embodiment, L is a moiety of a retinoid such as retinoic acid (RA), preferably all-trans-retinoic acid (ATRA), or an analog thereof.

In yet another embodiment, L is a moiety of a chemotherapeutic drug such as, but not limited to, 5-fluoro-uracil, adriamycin, or gefitinib (a small tyrosine kinase inhibitor, also known as ZD1839 or Iressa™, trade mark of AstraZeneca).

The term “paramagnetic metal”, as used herein, denotes metal ions which have unpaired electrons and includes paramagnetic lanthanide metals such as Gd(III) ion (Gd³⁺), which has 7 unpaired electrons, and paramagnetic transition metal ions such as Fe(III) ion (Fe³⁺), which has 4 unpaired electrons.

As used herein, “hydrocarbylene” for X₁ means a divalent radical derived from a hydrocarbyl radical, wherein said hydrocarbyl radical is a saturated or unsaturated C₂-C₁₀ aliphatic or C₃-C₁₀ cyclic radical, or a C₆-C₁₀ aromatic radical. When X₁ is an aliphatic chain, it is preferably a straight chain. When X₁ is an unsaturated aliphatic chain, it may contain one or more double and/or one or more triple bonds. Thus, for example, X₁ may be an alkylene, alkenylene, alkynylene, alkadienylene, alkadiynylene, cycloalkylene, phenylene or naphthylene radical or combinations thereof such as alkylphenyl, alkenylphenyl, alkynylphenyl, and the like. Examples of such divalent radicals include, without being limited to, vinylene, propenylene, butenylene, pentenylene, hexenylene, ethynylene (also called ethynediyl), propynylene, butynylene, pentynylene, hexynylene, cyclohexylene, phenylene, benzyl, ethylphenyl, vinylphenyl, ethynylphenyl, and the like. In a preferred embodiment, X₁ is a C₂ unsaturated chain, most preferably the radical —C≡C—. In another preferred embodiment, X₁ is phenylene.

In one embodiment of the invention, X₁ is a saturated or unsaturated aliphatic chain of 2-10 carbon atoms that may be optionally interrupted by at least one atom or radical selected from the group consisting of —O—, —S—, —N(R₅)—, —CO—N(R₅)—, —N(R₅)—CO—, —COO—, —OOC—, —N═N—, —C═N—, —N═C—, and/or X₁ is optionally substituted by halogen, —OR₅, —SR₅, epoxy, epithio, oxo or —COOR₅, wherein R₅ is H or (C₁-C₄) alkyl. In another embodiment, X₁ is phenylene that may be substituted by halogen, (C₁-C₄) alkyl, —OR₅, —SR₅, or —COOR₅.

As used herein, the term “C₁-C₄ alkyl” typically refers to a straight or branched alkyl radical having 1-4 carbon atoms and includes methyl, ethyl, n-propyl, isopropyl, n-butyl, isobutyl, sec-butyl and tert-butyl. As used herein, the term halogen refers to fluor, chloro, bromo, and iodo.

When X₂ is a covalent bond, the linker X₁ is linked directly to the N atom of the metal binding moiety or to a carbon atom of the monocyclic or polycyclic ring formed by the N atom, the two adjacent carbon atoms and other carbon and/or heteroatoms represented by the dotted line. When X₂ is phenylene, the linker X₁ is linked to a carbon atom of the phenyl radical and the phenyl radical, preferably at the para position, is linked to the N atom of the metal binding moiety or to a carbon atom of the monocyclic or polycyclic ring formed by the N atom, the two adjacent carbon atoms and other carbon and/or heteroatoms represented by the dotted line.

In one preferred embodiment of the invention, X₁ is —C≡C— or phenylene and X₂ is a covalent bond. In another preferred embodiment, X₁ is —C≡C— or phenylene and X₂ is phenylene.

The conjugates of the invention of the formula I, II or III comprising a metal binding moiety wherein at least three of R₁ to R₄ are —CH₂R₇ and R₇ is —COO⁻, —PO₃² or —P(R₉)O₂⁻, are designed to coordinate lanthanide metals with very high association constant, whereas the conjugates of the formula I, II and III comprising a metal binding moiety wherein each of R₁ to R₄ is H, (C₁-C₄) alkyl or —CH₂R₇ and R₇ is —COO⁻, —PO₃²⁻ or —P(R₉)O₂—, are designed to strongly coordinate transition metals.

In the conjugate of formula I, II or III, when R₁ to R₄ is —CH₂R₇, R₇ is preferably COOR₈, wherein R₈ is preferably C₄ alkyl, more preferably t-butyl, or R₇ is COO⁻ in the complex with the lanthanide metal.

In one embodiment of the invention, the N atom of the metal binding moiety of the conjugate may form, together with the two adjacent carbon atoms and further carbon and/or heteroatoms represented by the dotted line, a monocyclic or polycyclic ring system that may be saturated, unsaturated or aromatic. The monocyclic heteroring ring is preferably a 5-6 membered saturated or aromatic ring that may contain further O, S and/or N atoms, and is for example pyrrolidine, pyrrole, imidazole, oxazole, thiazole, piperidine, pyridine, or pyrimidine. In a most preferred embodiment, the monocyclic ring is the pyridine ring. In another embodiment, the ring is a polycyclic ring in which one or more rings may be carbocyclic such as quinoline or acridine. In a more preferred embodiment, the polycyclic ring is an acridine ring condensed with the macrocyclic ring of conjugate III.

According to one preferred embodiment of the invention, the conjugate is a conjugate I selected from the group consisting of formulas Ia to Ie:

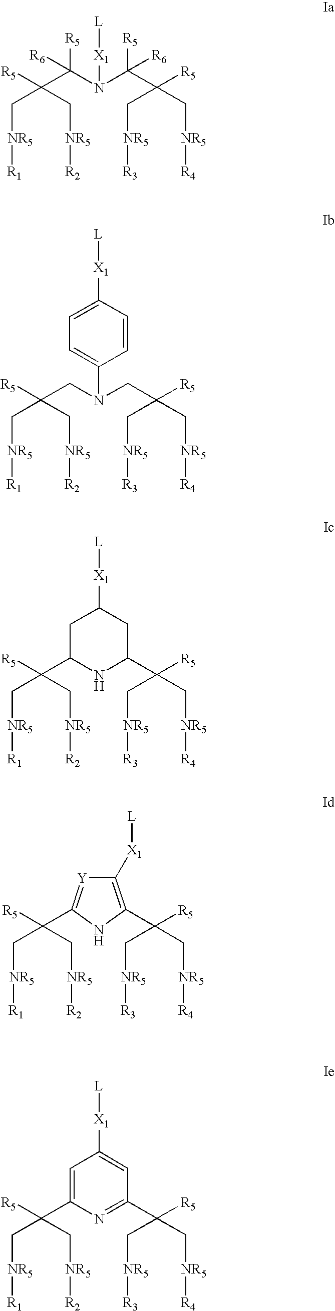

wherein

L, X₁, R₁ to R₆ are as defined above, Y is C, S, or N, and complexes thereof with paramagnetic lanthanide metals such as Gd(III), Eu(III), Dy(III) Tb(III), Tm(III), Yb(III) or Pr(III) or with paramagnetic transition metals such as Mn(II), Co(III), Ni(III), Fe(II) or Fe(III).

According to another preferred embodiment, the invention provides a conjugate II selected from the group consisting of formulas IIa to IIe:

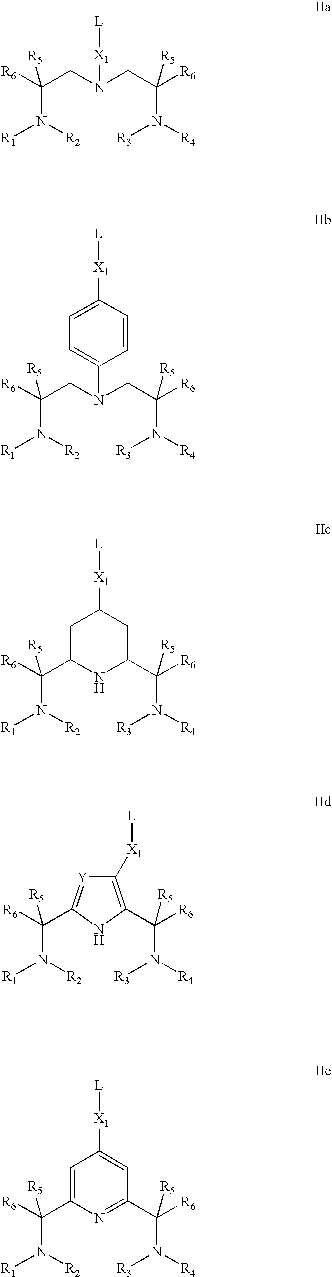

wherein L, X₁, Y, R₁ to R₆ are as defined above, and Y is C, S, or N; and complexes thereof with paramagnetic lanthanide metals such as Gd(III), Eu(III), Dy(III) Tb(III), Tm(III), Yb(III) or Pr(III) or with paramagnetic transition metals such as Mn(II), Co(III), Ni(III), Fe(II) or Fe(III).

In still another preferred embodiment, the invention provides a 1,4,7,10-tetraazacyclododecane conjugate III of formulas IIIa to IIIf:

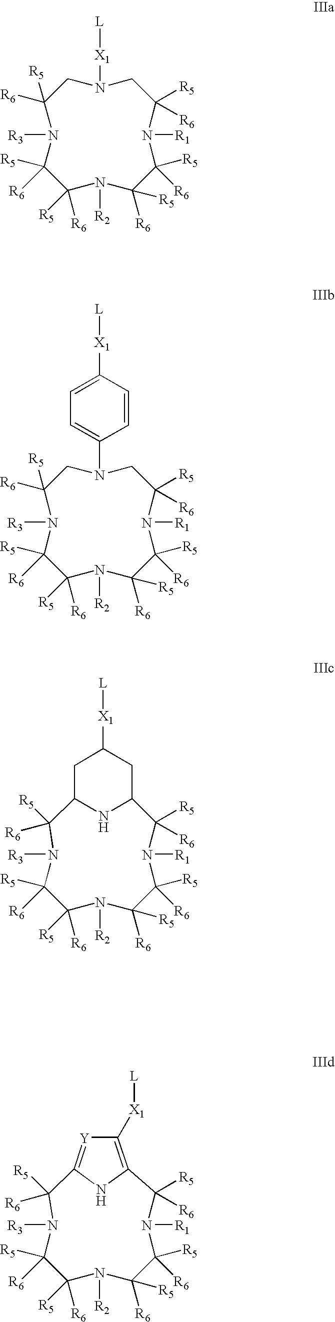

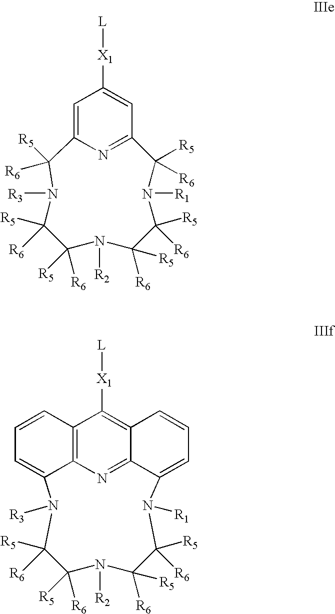

wherein, L, X₁, R₁ to R₆ are as defined above, Y is C, S, or N, and complexes thereof with paramagnetic lanthanide metals such as Gd(III), Eu(III), Dy(III) Tb(III), Tm(III), Yb(III) or Pr(III) or with paramagnetic transition metals such as Mn(II), Co(III), Ni(III), Fe(II) or Fe(III).

In one preferred embodiment of the invention, the conjugate is selected from the group consisting of conjugates of the formulas Ia to le wherein L is an ER ligand, more preferably, 17β-estradiol or tamoxifen, X₁ is —C≡C—, R₁ to R₃ each is —CH₂R₇, R₄ is H or (C₁-C₄) alkyl and R₇ is as defined above, more preferably, COOH. In a more preferred embodiment, the invention relates to the complexes of said conjugates, wherein R₇ is —COO⁻, with the lanthanide metals Gd(III), Dy(III), Eu(III), Tb(III), more preferably, Gd(III), for use as ER-specific MRI contrast agents. Since these metal ions have nine binding sites, the derived metal complexes of the octadentate conjugate will have a desirable extra binding site for a water molecule, which is desirable for MRI.

More preferably, the conjugate has the formula Ie, wherein R₁ to R₃ each is —CH₂COOR₈, R₄ is H or (C₁-C₄) alkyl, R₅ and R₆ each is H, R₈ is H or (C₁-C₄) alkyl, X₁ is —C≡C—, and L is 17β-estradiol or tamoxifen. Still more preferably, the invention provides a complex of the conjugate of formula Ie, wherein L is 17β-estradiol or tamoxifen, R₁ to R₃ each is —CH₂COO⁻, R₄, R₅ and R₆ each is H, with Gd(III), Tb(III) or Eu(III), preferably Gd(III).

In another preferred embodiment, the conjugate has the formula Ie wherein L is an ER ligand, more preferably, 17β-estradiol or tamoxifen, X₁ is —C≡C— and R₁ to R₄ each is H, (C₁-C₄) alkyl or —CH₂R₇ and R₇ is as defined above. These conjugates are designed to bind the transition Mn(II), Co(III), Ni(III), Fe(II) and Fe(III) metals to form ER-specific MI contrast agents. Since the preferred geometry of e.g. Fe(III) is octahedral, upon coordination of the pentadentate conjugate, a free site will be available for the binding of a water molecule, which is desirable for MRI.

More preferably, the conjugate has the formula Ie, wherein R₁ to R₆ each is H, and L is 17β-estradiol or tamoxifen. Still more preferably, the invention provides a complex of the conjugate of formula Ie, wherein L is 17β-estradiol or tamoxifen, R₁ to R₅ each is H with Mn(II), Co(III), Ni(III), Fe(II) and Fe(III), preferably, Fe(III).

In another preferred embodiment of the invention, the conjugate has the formula Ie, wherein L is an ER ligand, R₁ to R₄ each is —CH₂R₇, and R₇ is as defined above, designed to generate ER-specific luminescent lanthanide metal complexes. All nine coordination sites of the metal are tightly occupied and water coordination is not possible. The flexible ligand is likely to provide effective metal shielding. Water coordination is undesirable for luminescence applications since it can severely deactivate the metal emissive states by vibrational energy transfer. The aromatic pyridyl unit can serve as an efficient “antenna”, i.e. transfers excitation energy to the metal, which thereby becomes excited to the emissive state. In a preferred embodiment, in the conjugate of formula Ie, R₁ to R₄ each is —CH₂COOR₈, R₅ and R₆ each is H, and R₈ is H or (C1-C4) alkyl, and L is 17β-estradiol or tamoxifen. In another preferred embodiment, the invention provides a complex of said conjugate of formula Ie wherein R₁ to R₄ each is —CH₂COO⁻, with Tb(III) or Eu(III).

The conjugates of formula Ie and complexes thereof can be prepared starting from the synthesis of compound 17 herein below, in analogy to the synthesis described in Grohmann and Knoch (1996), starting with the 4-bromo-pyridine derivative. The next stepped are carried out similarly to the synthesis of compounds 6 and 15, depicted in Schemes I and II and fully described in Examples 1 and 2 herein.

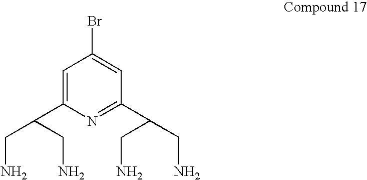

In another preferred embodiment of the invention, the conjugate is selected from the group consisting of formulas IIa to IIe, wherein L is an ER ligand, conjugated to a metal binding moiety designed to have seven binding sites. After binding the paramagnetic Gd(III), Tb(III) or Eu(III), two open coordination sites are available for binding water molecules for enhanced efficiency and sensitivity as ER-specific MRI contrast agents.

In a more preferred embodiment, in said conjugate Ia to IIe, L is 17β-estradiol or tamoxifen, X₁ is —C≡C—, R₁ to R₄ each is —CH₂R₇ and R₇ is as defined above. In a still more preferred embodiment, the conjugate is of formula IIe, wherein R₁ to R₄ each is —CH₂R₇, R₅ and R₆ each is H, R₇ is —COOR₈, and R₈ is H or (C₁-C₄) allyl. In most preferred embodiments, the conjugate is of formula Ile, wherein L is 17β-estradiol, X₁ is —C≡C—, R₁ to R₄ each is —CH₂COO-tBut, and R₅ and R₆ each is H, herein identified as compound 5, or L is tamoxifen, X₁ is —C≡C—, R₁ to R₄ each is —CH₂COO-tBut, and R₅ and R₆ each is H, herein identified as compound 14.

In another most preferred embodiments, the conjugate is of formula Ile wherein L is 17β-estradiol, X₁ is —C≡C—, R₁ to R₄ each is —CH₂COOH, and R₅ and R₆ each is H, herein identified as compound 6, or L is tamoxifen, X₁ is —C≡C—, R₁ to R₄ each is —CH₂COOH, and R₅ and R₆ each is H, herein identified as compound 15.

In still another most preferred embodiment, the conjugate of formula Ile, wherein L is 17β-estradiol or tamoxifen, R₁ to R₄ each is —COO and R₅ and R₆ each is H, is complexed with a paramagnetic lanthanide-metal selected from the group consisting of Gd(III), Tb(III) and Eu(III). Most preferably, the conjugate comprises 17β-estradiol and is complexed to Gd(III), herein identified as compound 7, or comprises tamoxifen and is complexed to Gd(III), herein identified as compound 16.

The conjugates of formula Ile and metal complexes thereof can be obtained by a multi-step synthesis as depicted in Scheme I and fully described in Example 1 herein. First, an alkyl ester of the metal binding moiety is synthesized starting from 4-hydroxy-2,6-pyridinedicarboxylic acid, the alkyl ester is then coupled with an ER ligand, for example 17α-ethynylestradiol, using Pd(II)/Cu(I) as catalyst in analogy to the procedure described in Jackson et al, 2001, to obtain the alkyl, e.g., t-butyl, ester of the conjugate, as represented by compound 5. In the next step, the ester is hydrolyzed to give the free acid, as represented by compound 6, which is then complexed with the lanthanide paramagnetic metal, e.g., Gd(III), to give the complex, as represented by compound 7. The multi-step synthesis of compounds 14, 15 and 16 is depicted in Scheme II and fully described in Example 2 herein.

In another preferred embodiment of the invention, the conjugate is selected from the group consisting of formula IIIa to IIIf, wherein X₁ is —C≡C— and L is 17β-estradiol or tamoxifen.

In a more preferred embodiment, the conjugate is of formula IIIe, wherein R₁ to R₃ each is —CH₂R₇, R₅ and R₆ each is H, R₇ is —COOR₈, and R₈ is H or (C₁-C₄) alkyl, more preferably wherein R₁ to R₃ each is —CH₂COOH, R₅ and R₆ each is H and L is 17β-estradiol or tamoxifen. In another preferred embodiment, the conjugate of formula IIIe, wherein L is β-estradiol or tamoxifen, R₁ to R₃ each is —CH₂COO⁻ and R₅ and R₆ each is H, is complexed with a paramagnetic lanthanide-metal selected from the group consisting of Gd(III), Tb(III) and Eu(III), preferably Gd(III).

In another preferred embodiment of the invention, the conjugate is of formula IIIf, wherein R₁ to R₃ each is —CH₂R₇, R₅ and R₆ each is H, R₇ is —COOR₈, and R₈ is H or (C₁-C₄) alkyl, more preferably wherein R₁ to R₃ each is —CH₂COOH, R₅ and R₆ each is H and L is 17β-estradiol or tamoxifen. In another preferred embodiment, the invention provides a complex of the conjugate IIIf, wherein L is 17β-estradiol or tamoxifen, R₁ to R₃ each is —CH₂COO⁻ and R₅ and R₆ each is H, with a paramagnetic lanthanide-metal selected from the group consisting of Gd(III), Tb(III) and Eu(III), preferably Eu(III).

The conjugates of formula IIIf can be obtained by first synthesizing the pyridino-porphyrine derivative represented e.g. by compound 18, starting from 4 bromo-2,6-bis(chloromethyl)pyridine, as depicted in Scheme III. The next steps in the synthesis of conjugates of formula IIIf are analogous to those described for compounds 6 and 15.

The complexes of the conjugates of formulas I, II, and II hereinabove having a —COOR₈, —COO—, PO₂(R₈)(R₉), PO₃²⁻, PO₃H₂ or —P(R₉)O₂⁻ group with a lanthanide paramagnetic metal selected from the group consisting of Gd (III), Eu(III), Dy(III), Tb(III), Tm(III), Yb(III) and Pr(III) and complexes of the conjugates of formulas I and II having an amine group with a paramagnetic transition metal selected from the group consisting of Mn(II), Co(III), Ni(III), Fe(II) and Fe(III), are magnetic resonance imaging (MRI) sensitive and are suitable for use as MRI contrast agents, particularly to indicate the presence of receptors in tissues, more particularly, malignant tissues, and to monitor drug delivery by means of non-invasive molecular MRI. The molecular imaging approach according to the invention is thus highly useful for specific detection and diagnosis of cancerous tumors or other abnormalities marked by high levels of specific receptors, such as breast and prostate cancer, and for prognosis of treatment and assessment of the resistance of such tumors to chemotherapeutic treatment.

As used herein, the terms “MRI contrast agent”, “MRI probe” and “probe” are used interchangeably and are all intended to refer to the metal complexes of the conjugates of present invention.

Thus, in another aspect, the present invention provides a quantitative and non-invasive method to evaluate the level of receptors by means of molecular MRI. This novel molecular imaging approach has the capacity to tremendously improve the detection, diagnosis and evaluation of prognosis of cancers, such as breast and prostate cancer.

The novel MRI sensitive metal complexes of the ligand conjugates provided by the invention can bind specifically to receptors, through the receptor ligand moiety, and are useful to identify the presence of the receptors by non-invasive MRI methods through the second functional group, i.e. the metal binding moiety complexed to the lanthanide or transition metal.

In one preferred embodiment, this molecular imaging approach is applied to determination of the level and spatial distribution of estrogen receptor and is highly useful for specific detection, diagnosis and prognosis evaluation of cancers, particularly breast and prostate cancer. For this purpose, novel molecules synthesized according to the invention have an ER ligand moiety and bind specifically to ER-α, and said estrogenic function is tagged with a Gd chelate (herein sometimes referred to as “ER-ligand-Gd”), that changes the MRI signal in the binding site. The biological activity and MRI detectability of these novel molecules were determined in vitro on isolated recombinant ER-α and on whole ER⁺ human breast cancer cell lines, and in vivo in orthotopic ER⁺ implanted tumors.

The ERα-specific contrast agents of the present invention (compounds 7 and 16) selectively bind to the ER-α and thus enhance the MRI signal in its vicinity. It is shown herein in the examples that the Gd complex 7, that contains the 17β-estradiol moiety, induces proliferation of the ER⁺ MCF7 and T47D human breast cancer cells in a dose dependent manner as 17β-estradiol, although at different doses (˜30 fold higher), but still in the pharmacological range of micro-molar. Compound 7 further exhibited strong binding affinity to ER, comparable to that of tamoxifen.

Both the ER-ligand-Gd and the gadolinium free precursor (herein sometimes designated “ER-ligand”) induced the growth of a range of ER⁺ human breast cancer cells, demonstrating binding and activation of the estrogen receptor in a time and dose dependent manner. The induction of proliferation occurred at a dose of nanomolar for the ER-Ligand 6 and micromolar for ER-ligand-Gd 7. The ER-ligand-Gd 7 demonstrated high relaxivity. It was also non-toxic in immunodeficient mice.

MRI monitoring of the inner organs and orthotopic ER⁺ MCF7 tumors in immunodeficient mice during 24 hours after the iv administration of ER-ligand-Gd 7 revealed the variable distribution in the body, the pharmacokinetics, and the clearance through the kidneys into the bladder. Selective residual presence of ER-Ligand-Gd 7 was detected in the tumor 24 hours after its intravenous (iv) bolus administration. In other body organs, except the bladder, no residual ER-Ligand-Gd 7 was detected. Overall, the results indicate that the novel compound ER-ligand-Gd 7 is a good candidate for developing MR molecular imaging of the estrogen receptor.

It is further shown herein that MRI signal enhancement in the tumors due to administration of 7 was persistent throughout the experiment time. The slow entrance and clearance of 7 is thus attributed to its entrance to the cells and binding to the ER. To detect the binding of 7 to ER, MRI measurements were performed 24 hours after its administration, when all other tissues appeared to be cleared from 7. Using a dose of 0.024 mmol/kg it was found according to the invention that the ER positive breast tumors still exhibited small but persistent increase in T₁ relaxation rate, while in the muscle this rate returned to the pre administration value. This increase suggests binding of 7 to the high level of ERs present in the nuclei of the cells. High level of ER in breast tumor cells was confirmed by immunostaining of the receptor.

The MRI contrast agent of the invention may be administered to a patient intravenously as a high or low dose bolus injection, or the high dose may be administered under a slow infusion protocol, hereinafter sometimes referred to as a “drip” protocol, for example, over 60 minutes. The drip protocol enables to reach a steady state, which is important, for example, to test the effectiveness of drug delivery.

Thus, in another aspect, the present invention provides an MRI contrast agent comprising a metalated conjugate of the invention of the formula I, II or III, that can bind to a receptor and enhance the MRI signal in its vicinity. In one most preferred embodiment, the receptor is ER and the metalated complex selectively binds to the ER and enhances the MRI signal in its vicinity. This probe is non-toxic and is potent both as a ligand of ER that induces proliferation and as a sensitive MRI contrast agent for mapping the receptor in vivo. Therefore, use of such a probe in humans opens the way for molecular imaging of the breast for improved diagnostic and prognostic purposes. Other steroid receptors that are implicated in breast cancer, e.g. progesterone receptor, or in other cancers, e.g. androgens in prostate cancer, can be mapped and evaluated according to the present invention. Hence, is an object of the invention to extend the diagnostic means of cancer to non-invasive molecular MRI.

In another aspect, the invention relates to a method of using a contrast agent to obtain magnetic resonance images of a patient comprising: administering to the patient a MRI contrast agent comprising a metalated complex of the formula I, II or III, and taking magnetic resonance images of the patient prior to said administration and thereafter.

In a further aspect, the invention provides a molecular MRI method comprising the steps of:

(i) administering to a patient a MRI contrast agent comprising a paramagnetic metal complex of a conjugate of formula I, II or III as defined hereinabove; and

(ii) subjecting the patient to magnetic resonance imaging by generating at least one MR image of the target region of interest within the patient's body prior to the administration of the MRI contrast agent said bolus intravenous injection and at least one MR image thereafter.

The MRI contrast agent is dissolved in a suitable buffer and administered either as a bolus intravenous injection or as a slow infusion (drip protocol). The first MR image of the target region of interest, e.g. the region of a tumor, is generated prior to the administration (time t₀), and one or more MR images are generated at one or more times after administration (time t, e.g., t₁, t₂, t₃, etc.), The presence of the paramagnetic probes in the cells or tumors is expected to affect T₁ and T₂ nuclear relaxation rates, as well as to evoke a susceptibility T₂* effect. We have estimated that in MCF7 tumors, the expected changes due to the presence of an MRI probe bound to ER would be about 5-10% in T₁ and 10-17% in the susceptibility effect (T₂*) (Kenna et al., 1994; Weisskoff et al., 1994; Boxerman et al., 1995; Dennie et al., 1998).

In the methods of the invention, the MR images of the target region of interest (ROI) at time t₀ and thereafter at time t may be T₁-weighted, T₂-weighted, T₂*-weighted or an actual experimental parameter (e.g. variable inversion times) images obtained using standard protocols. For generating T₁-weighted images, T₁-weighted, 3D gradient echo with a flip angle, e.g. 30°, may be employed or a 2D inversion or saturation recovery sequence with varying inversion or saturation times ranging from e.g., 10 to 10,000 msec, and a gradient echo acquisition. The latter sequence enables mapping the T₁ relaxation time. Complexed sequences based on susceptibility gradient echo and T₂ weighted spin echo protocols can be carried out to generate the T₂*- and T₂-weighted MR images, respectively, as well as sequences for measuring T₂* and T₂ that employ variable echo times in gradient and spin echo, respectively.

In one preferred embodiment, the target ROI in the patient's body is the region of a suspected tumor, and the MR images are taken at time zero (t₀) and at at least a second time point (t₁) or at plural sequential time points after the injection of the contrast agent, which is determined depending on the purpose of the MRI measurement. For diagnosis of tumors, follow-up of chemotherapeutic or hormonal treatment of a malignant tumor and prognosticating the effectiveness of chemotherapy or hormonal treatment of a malignant tumor, it is important to wait until most of the contrast agent has been cleared from the patient's body, apart from detectable amounts, which remain bound to the receptors of a suspected tumor. As shown herein in mice, almost complete clearance of the contrast agents of the invention takes place 8 to 24 after their administration. In humans, the clearance might be faster. Thus, according to the invention, the second, third, fourth and additional MRI measurements following administration of the contrast agent are preferably performed within 1, 2, 3, 4 and up to 24 hours after administration.

For monitoring drug delivery of a chemotherapeutic drug to a malignant tumor, it is important to perform the second or more measurements while most of the contrast agent is still in the patient's body, in order to assess whether the drug has reached the tumor or not. Thus, for assessment of the efficiency of delivery using the drip protocol, the optimum timing for obtaining the second image is when the concentration in the blood reaches a steady state, within 1-4 hours after administration of the probe.

The data obtained at time t₀ and at time t are processed, for example, to generate a color coded map selected from the group consisting of enhancement, reduction, T₁ relaxation rate, T₂ relaxation rate and T₂* relaxation rate map, on a pixel by pixel basis or on the basis of selected regions of interest (ROIs), and the processed data is then analyzed.

In one preferred embodiment of the invention, the MRI method is applied for tumor diagnosis of a patient suspected of having a tumor. The MRI contrast agent comprising a metal complex of a conjugate of formula I, II or III wherein L is a ligand of a receptor associated with said tumor is administered to the patient, MR images are acquired prior to administration and thereafter, the data generated is processed and analyzed for the presence or absence of a tumor.

In another preferred embodiment, the MRI method is applied for prognosticating the effectiveness of a chemotherapeutic or hormonal treatment of a patient bearing a malignant tumor and being treated with a chemotherapeutic or hormonal agent. The MRI contrast agent comprising a metal complex of a conjugate of formula I, II or III wherein L is a ligand of a receptor associated with said tumor is administered to the patient, MR images are acquired prior to administration and thereafter, the data generated is processed, and the analysis of the processed data will enable prognosis of the effectiveness of the chemotherapeutic or hormonal agent in the treatment of said tumor in said patient.

In yet another preferred embodiment, the MRI method is applied for follow-up of malignant tumor therapy in a patient by a chemotherapeutic or hormonal agent. The MRI contrast agent comprising a metal complex of a conjugate of formula I, II or III wherein L is a ligand of a receptor associated with said tumor is administered to the patient, MR images are acquired prior to administration and thereafter, the data generated is processed, and the analysis of the processed data will enable evaluation of the effectiveness of the chemotherapeutic or hormonal agent in the treatment of said tumor in said patient.

In still another preferred embodiment of the invention, the MRI method is applied for monitoring a chemotherapeutic drug or an anti-hormonal agent delivery to a malignant tumor. The MRI contrast agent comprising a metal complex of a conjugate of formula I, II or III wherein L is a chemotherapeutic drug or an anti-hormonal agent that binds to a receptor associated with said tumor is administered to the patient, MR images are acquired prior to administration and thereafter, the data generated is processed, and the analysis of the processed data will enable evaluation of the effectiveness of the efficiency of delivery and entrance of the chemotherapeutic or anti-hormonal agent which determines the treatment of said tumor in said patient.

In preferred embodiments of the invention, the malignant tumor is breast cancer or prostate cancer

In one preferred embodiment of the invention, the MRI contrast agent is the Gd(III) complex herein designated compound 7. In another preferred embodiment, the MRI contrast agent is the Gd(III) complex herein designated compound 16.

In another aspect, the invention relates to the use of any of the conjugates defined above and a paramagnetic transition metal or lanthanide metal selected from the group consisting of Mn(II), Ni(III), Fe(II), Fe(III), Co(III) or Gd(III), Tb(III), Dy(III), Eu(III), Tm(III), Yb(III) and Pr(III), respectively, for the preparation of a contrast agent for MR imaging for the purpose of tumor diagnosis, prognosticating the effectiveness of hormonal and chemo-therapy in the treatment of cancer, for follow-up of cancer therapy or monitoring anti-hormone or chemotherapeutic drug delivery to a tumor. In a most preferred embodiment, the MRI contrast agent is the Gd(III) complexed compound 7 or compound 16 applied for MR imaging of breast cancer.

In conclusion, according to the present invention, multistep synthetic procedures were developed and applied in order to obtain highly purified novel estrogen receptor ligands with MRI tagged paramagnetic center. These paramagnetic estrogen receptor ligands are candidates as molecular probes for mapping the estrogen receptor in vivo using contrast enhanced MRI. The first prototype, based on estradiol and containing an MRI tag of a pyridinium-Gd complex (Compound 7), was found to be agonistic in its hormonal activity, non toxic in mice and rats, and sufficiently potent both as a ligand of ER and a sensitive MRI contrast agent. The second prototype, based on tamoxifen and the same MRI tag as above (Compound 16), was found to act as an antagonist to estrogen and in a similar manner to tamoxifen. Both ligands were found to bind to ER at the micromolar range and enhance the water relaxation rates in their vicinity by more than an order of magnitude. This enhancement may increase upon binding to the receptor.

The invention will now be illustrated by the following non-limiting examples.

### EXAMPLES

### Example 1

### Synthesis of the 17β-estradiol-17α-ethynyl-pyridine Conjugate Compound 6 and its Gd Complex Compound 7

The steps for the synthesis of compound 6 and 7 are shown in Scheme I, just before the References.

### 1.1 Synthesis of Intermediate 1 [Diethyl 4-bromo-2,6-pyridinedicarboxylate]

Intermediate 1 was reported by Takalo and Kankare (1987). The following is a modified procedure: To a vigorously stirred solution of Br₂ (24 gm) in petroleum ether (100 ml, b.p. 40-60° C.), PBr₃ (48 gm) was added. After stirring for a few minutes at room temperature, the resulting PBr₅ was washed several times with petroleum ether by decantation and dried in vacuo. 4-hydroxy-2,6-pyridinedicarboxylic acid (10 gm) was added to the same reaction vessel and, after thorough mixing, the temperature of the bath was raised to 90° C. and maintained at that temperature for 3 hrs. The cooled mixture was stirred with CHCl₃ (75 ml) and filtered through a Whatman filter paper. Absolute ethanol (200 ml) was added to the filtrate in small portions and the solution was concentrated in vacuo, until it looked a little bit viscous. Immediately after, when it was still hot, while evaporating much of the ethanol in rotary-evaporator at 60° C. to make it concentrated, small pieces of ice were added slowly into it until white fluffy Intermediate 1 started separating out. The crystallization of Intermediate 1 from the mother liquor was even better and faster when the concentrated solution, after adding few pieces of ice, was seeded with 10 mg of Intermediate 1 prepared before. For the completion of this process it was left in the freezer at 0° C. for one night and next day morning it was filtered off under vacuum. Yield: 8 gm.

### 1.2 Synthesis of Intermediate 2 [4-bromo-2,6-bis(hydroxymethyl)pyridine

Intermediate 2 was reported by Takalo et al. (1988). The following is a modified procedure: Sodium borohydride was added in small portions to a suspension of Intermediate 1 in absolute ethanol (which was previously dried and distilled from Mg(OEt)₂) over a period of 0.5 hrs. After stirring for 2 hrs at room temperature the mixture was heated under reflux for 15 hrs and evaporated in vacuo. A saturated solution of NaHCO₃ was added to the residue, the solution was brought to boiling and boiled for 5 minutes and then few pieces of ice (equivalent to the amount of water as said in Takalo et al., 1988) were added and the mixture was immediately put in the freezer. The mixture was allowed to stand overnight in the freezer and the white precipitate filtered off under vacuum next day morning. It was left for air-drying for 2 hrs and then the precipitate was taken in a thimble and extracted continuously with acetone in a Soxhlet apparatus for 24 hrs. The acetone solution was evaporated to get Intermediate 2 in desired yield.

### 1.3 Synthesis of Intermediate 3 [4-bromo-2,6-bis(bromomethyl)pyridine

This compound was prepared from Intermediate 2 using PBr₃ and CHCl₃ according to the procedure disclosed in Takalo et al. (1988). Care was taken for proper neutralization with 5% NaHCO₃. The pH of the reaction mixture was checked with litmus paper all the time.

### 1.4 Synthesis of Intermediate 4 [4-bromo-2,6-bis[N,N-bis(t-butoxycarbonylmethyl)aminiomethyl]pyridine]

This compound was prepared from Intermediate 3 by reaction with the secondary amine HN(CH₂COOt-But)₂, Na₂CO₃ and CH₃CN, as described by Takalo et al. (1988) (see Scheme I).

### 1.5 Synthesis of Compound 5

Intermediate 4 (6.6125 gm, 9.84 mmol) obtained in 1.4 above, 17α-ethynyl-estradiol (2.912 gm, 9.84 mmol), Pd(PPh₃)₂Cl₂ (0.137 gm, 0.19 mmol) and CuI (0.0746 gm, 0.397 mmol), were placed under a nitrogen atmosphere, and dry degassed THF (52 ml) was added followed by diisopropylamine (Prⁱ₂NH) (52 ml). The mixture was stirred under a nitrogen atmosphere in the dark at room temperature for 24 hrs. The brown mixture was filtered and reduced to dryness in vacuo to give the crude product. The glassy looking crude product was purified by a slow dropping column using Merck Kieselgel 60 silica (0.063-0.200 min) and hexane/ethyl acetate as an eluant, yielding Compound 5 as a fluffy yellow solid (4.25 gm, 48.66%).

¹HNMR (400 MHz, CDCl₃): δ 10.02 (s, 1H, phenolic-OH), 7.53 (s, 2H, pyridyl), 7.12 (d, 1H, J=8.5 Hz, H1), 6.61 (dd, 1H, J1=8.2 Hz, J2=2.7 Hz, H2), 6.56 (d, 1H, J=2.4 Hz, H4), 5.23 (s, 1H, β-hydroxyl), 3.99 (s, 4H, H24 methylene), 3.42 (s, 8H, H25 methylene), 2.80 (m, 2H, H6), 2.37 and 2.09 (m, 4H, H15/H16), 2.37 (m, 1H, H9), 1.85 and 1.44 (m, 2H, H11), 1.87 and 1.33 (m, 2H, H7), 1.81 (m, 2H, H12), 1.78 (m, 1H, H8), 1.70 (m, 1H, H14), 1.51 (s, 36H, H27 methyl), 0.92 (s, 3H, H18 methyl). ¹³CNMR (400 MHz, CDCl₃): δ 171.92 (C26), 160.66 (C21), 154.91 (C3), 139.61 (C5), 133.85 (C10), 133.70 (C23), 127.97 (C1), 124.54 (C22), 116.65 (C4), 114.11 (C2), 98.34 (C17), 85.91 (C27), 82.53 (C19), 81.82 (C20), 60.97 (C24), 57.33 (C25), 51.18 (C14), 49.07 (C13), 44.81 (C9), 40.90 (C16), 40.37 (C8), 34.54 (C12), 31.08 (C6), 29.60 (C28), 28.56 (C7), 27.83 (C11), 24.38 (C15), 14.28 (C18). The NMR assignments were assisted by ¹³C-¹H correlation (HET-CORR) 2-D spectra.

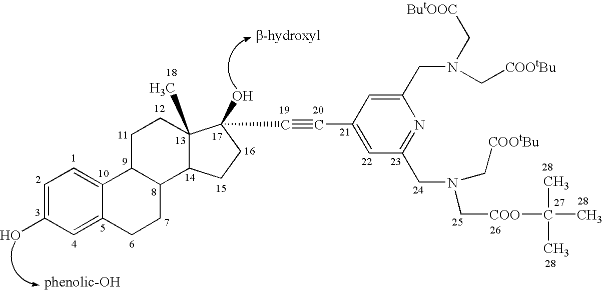

Best TLC of the reaction mixture for synthesis of Compound 5 dissolved in ethyl acetate was obtained in 55% EtOAc:hexane. Compound 5 came out in 40% EtOAc:hexane of a Merck Kieselgel 60 silica (0.063-0.200 mm) column.

### 1.6 Synthesis of Compound 6

The tetra-carboxylate Compound 5 (4.2494 gm, 4.7 mmol) obtained in 1.5 above was subjected to hydrolysis with trifluoroacetic acid. For this purpose, it was dissolved in excess of trifluoroacetic acid (120 ml) and vigorously stirred in an ice-bath for 1.5 hrs. The trifluoroacetic acid was evaporated in vacuo without heating. The residue was triturated with ether (3×100 ml) and filtered to give the tetra-carboxylic acid Compound 6 (2.3824 gm, 75%).

¹HNMR (400 MHz, DMSO-d₆): δ 9.01 (s, 1H, phenolic-OH), 7.46 (s, 2H, pyridyl), 7.05 (d, 1H, J=8.8 Hz, H1), 6.49 (dd, 1H, J1=8.3 Hz, J2=2.4 Hz, H2), 6.43 (d, 1H, J=2.3 Hz, H4), 5.72 (s, 1H, β-hydroxyl), 3.90 (s, 4H, H24 methylene), 3.45 (s, 8H, H25 methylene), 2.82 (m, 2H, H6), 2.42 and 2.09 (m, 4H, H15/H16), 2.42 (m, 1H, H9), 1.90 and 1.44 (m, 2H, H11), 1.94 and 1.34 (m, 2H, H7), 1.72 (m, 2H, H12), 1.67 (m, 1H, H8), 1.60 (m, 1H, H14), 0.85 (s, 3H, H18 methyl). ¹³CNMR (400 MHz, DMSO-d₆): δ 172.58 (C26), 159.08 (C21), 155.04 (C3), 137.38 (C5), 132.07 (C10), 130.50 (C23), 126.38 (C1), 122.78 (C22), 115.09 (C4), 112.92 (C2), 99.59 (C17), 82.66 (C19), 78.93 (C20), 58.94 (C24), 54.63 (C25), 49.67 (C14), 47.45 (C13), 43.43 (C9), 33.15 (C12), 30.87 (C6), 29.37 (C7), 27.13 (C11), 22.78 (C15), 13.02 (C18). C16, C8 merges with DMSO peak. The NMR assignments were assisted by ¹³C-¹H correlation (HET-CORR) 2-D spectra.

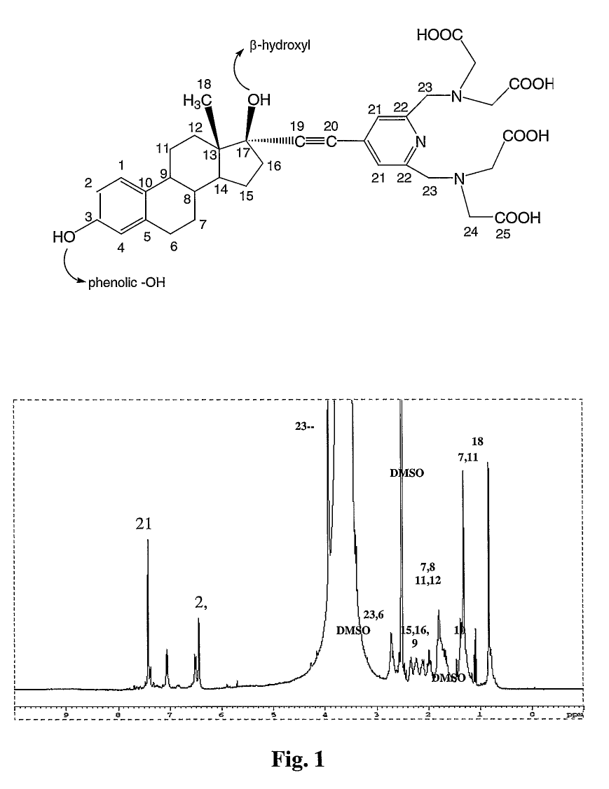

The ¹H NMR spectrum of the metal free Compound 6 is shown in FIG. 1. The conjugate was dissolved in DMSO and transferred to a 5 mm NMR tube. The spectrum was recorded at 400 MHz (Bruker, AMX-400) accumulating 100 transients with 90 degrees pulses, 3 sec repetition time, 16K data points and 8K Hz spectral width. A line broadening of 0.1 Hz was applied in the processing. The assignment of the main peaks is according to the numbering of protons in the chemical structure of the ER-ligand.

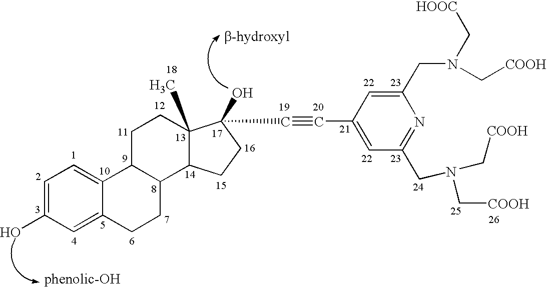

### 1.7 Synthesis of Compound 7

The tetraacid Compound 6 (1 gm, 1.508 mmol) obtained in 1.6 above, was dissolved in water (17 ml) and the pH was adjusted to 6.5 with solid NaHCO₃. Gd (III) chloride (0.437 gm, 1.659 mmol) in water (7 ml) was added over 15 min and the pH was maintained in the range of 5-7. After the mixture was stirred for 1.5 hrs at room temperature, the pH was raised to 8.5 with 1M NaOH and the precipitate was filtered off. Acetone was added to the filtrate to precipitate the Gd complex Compound 7 (0.739 gm, 46%). Compound 7 was filtered and washed with acetone.

IR (KBr pellet): 3425, 2925, 1602, 1406 cm⁻¹ ν (OH, C≡C, C═O and CO). Anal. Calcd for C₃₅H₃₇N₃O₁₀GdNa.6H₂O.2NaCl: C, 40.83, H, 4.80; N, 4.08. Found: C, 40.83, H, 4.90; N, 3.88.

### Example 2

### Synthesis of the Metaled Tamoxifen-Ethynylpyridine Conjugate Compound 15 and its Gd Complex Compound 16

The multi-steps synthesis of Compounds 15 and 16 is depicted in Scheme II.

### 2.1 Separation of E, Z isomers of Intermediate 10

The synthesis of Intermediates 8, 9 and 10 was carried out according to Hardcastle et al. (1995) and is shown in Scheme II.

The mixture containing the tamoxifen precursor Intermediate 10 obtained after column chromatography was dried under high vacuum and minimum volume of absolute ethanol was added to dissolve it. It was heated to boiling and then transferred to the freezer at 0° C. After 2 hrs it was brought out of the freezer and kept at room temperature. The sudden temperature difference makes the E-isomer to precipitate as fluffy white crystals that should be kept under constant supervision so as to filter the crystals off at the right moment before the Z-isomer also starts that has to be followed by ¹H NMR of the precipitated crystals. Sometimes the crystals of E-isomer also start falling out when it is in the freezer, thus the solution should be continuously monitored so that after the initiation of the crystallization process it can be brought out of the freezer and kept at room temperature. The total time taken for crystallization varies depending on the room temperature. At 30° C. it takes about 2-3 hrs and at low room temperature it may take 10-12 hours. Thus, it is preferable to heat the room at 30° C. for faster and efficient crystallization.

### 2.2 Synthesis of Intermediate 11 [4-Trimethylsilylethynyl-2,6-bis[N,N-bis(t-butoxycarbonylmethyl)aminomethyl]pyridine]

For the synthesis of Intermediate 11, Intermediate 4 (6.9292 gm, 10.3 mmol), trimethylsilyl acetylene (1.218 ml, 12 mmol), Pd(PPh₃)₂Cl₂ (0.144 gm, 0.2 mmol) and CuI (0.0193 gm, 0.1 mmol) were placed under a nitrogen atmosphere and dry Et₂NH (50 ml) was added. The mixture was stirred under a nitrogen atmosphere in the dark at room temperature for 8 hrs. The brownish yellow mixture was filtered and reduced to dryness in vacuo. The residue was taken up in benzene, washed several times with water, and dried over anhydrous Na₂SO₄. The benzene solution was evaporated to give the crude product. The crude product was purified by a slow dropping column using Merck Kieselgel 60 silica (0.063-0.200 mm) and hexane/ethyl acetate as an eluant to yield Intermediate 11 (2.99 gm, 42%) as a brown viscous oil.

¹H NMR (250 MHz, CDCl₃): δ 7.74 (s, 2H, pyridyl), 4.02 (s, 4H, methylene), 3.47 (s, 8H, methylene), 1.46 (s, 36H, ^{t}Bu), 0.24 (s, 9H, SiMe₃).

Best TLC obtained in 30% EtOAc:hexane and Intermediate 11 was separated by a slow dropping column that came in 15-20% EtOAc:hexane using Merck Kieselgel 60 silica (0.063-0.200 mm) column.

### 2.3 Synthesis of Intermediate 12 [4-ethynyl-2,6-bis[N,N-bis(t-butoxycarbonyl-methyl)aminomethyl]pyridine]

Intermediate 11 (2.99 gm, 4.34 mmol), obtained in 2.2 above was treated with a solution of Bu₄NF.3H₂O (0.3765 gm, 1.44 mmol) in THF (50 ml), at room temperature for 1 hr. The brown solution was reduced to dryness in vacuo and the residue was dissolved in ether. The ether layer was washed with water and dried over anhydrous Na₂SO₄. Ether was evaporated to give the crude product that yielded a pale cream solid Intermediate 12 (1.73 gm, 65%) when column chromatographed, using Merck Kieselgel 60 silica (0.063-0.200 mm) column and hexane/ethyl acetate as an eluant.

¹H NMR (250 MHz, CDCl₃): δ 7.65 (s, 2H, pyridyl), 3.99 (s, 4H, methylene), 3.46 (s, 8H, methylene), 3.24 (s, 1H, C≡CH) 1.46 (s, 36H, ^{t}Bu).

Best TLC obtained in 25% EtOAc:hexane and the compound with higher R.F. was Intermediate 12, which was separated by a slow dropping column using Merck Kieselgel 60 silica (0.063-0.200 mm) column that came in 17% EtOAc:hexane. The unreacted SiMe₃ derivative came out before Intermediate 12 in 15% EtOAc:hexane.

### 2.4 Synthesis of Intermediate 13

Intermediate 12 (1.23 gm, 1.9 mmol) and Intermediate 10 (the Z-isomer, see 2.1 above) (1.411 gm, 1.9 mmol), Pd(PPh₃)₂Cl₂ (0.063 gm, 0.09 mmol) and CuI (0.0037 gm, 0.02 mmol), were placed under a nitrogen atmosphere and dry Et₂NH (30 ml) was added. The mixture was stirred under a nitrogen atmosphere in the dark at room temperature for 3 hrs. The brown mixture was filtered and reduced to dryness in vacuo and the residue was taken up in ethyl acetate. The ethyl acetate layer was washed a few times with water, dried over anhydrous Na₂SO₄ and concentrated in vacuo to give the crude product. The crude product yielded a bright yellow solid Intermediate 13 (1.26 gm, 45%) after column chromatography using a Merck Kieselgel 60 silica (0.063-0.200 mm) column and hexane/ethyl acetate as an eluant.

¹H NMR (250 MHz, CDCl₃): δ 7.71 (s, 2H, pyridyl), 7.52 (d, 2H, J=7.3 Hz, H_{d}) 7.10-7.19 (m, 7H, H_{c}, Hₑ), 6.76 (d, 2H, J=9.1 Hz, H_{b}) 6.56 (d, 2H, J=8.8 Hz, Hₐ), 4.08-4.16 (m, 2H+4H, CH₂OAr+methylene), 3.71 (t, 2H, J=5.7 Hz, CH₂Cl), 3.53 (s, 8H, methylene), 2.47 (q, 2H, J=7.1 Hz, CH₃CH₂), 1.47 (s, 36H, ^{t}Bu), 0.88 (t, 3H, J=7.3 Hz, CH₃).

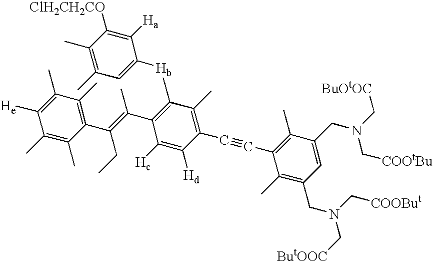

Best TLC in 27% EtOAc:hexane, the compound glowed brightly in UV light and was separated by a slow dropping column that came in 15-20% EtOAc:hexane. Polarity was later increased to 22% EtOAc:hexane to enable all of Intermediate 13 to come out of the column.

### 2.5 Synthesis of Compound 14

A mixture of Intermediate 13 (1.2557 gm, 1.2 mmol) and dimethylamine (33%, 20 ml) in EtOH was heated in a Fischer Potter at 100° C. for 90 min and then allowed to cool, poured into ether (100 ml), washed with water (3×100 ml), dried on anhydrous Na₂SO₄ and concentrated in vacuo. Flash column chromatography (ethyl acetate) using a Merck Kieselgel 60 silica (0.040-0.063 mm, 230-400 mesh) column gave Compound 14 (0.7059 gm, 56%) as a bright yellow fluffy solid.

¹H NMR (250 MHz, CD₂Cl₂): δ 7.62 (s, 2H, pyridyl), 7.53 (d, 2H, J=8.5 Hz, H_{d}), 7.26 (d, 2H, J=8.2 Hz, H_{c}), 7.14-7.22 (m, 5H, Hₑ), 6.80 (d, 2H, J=8.5 Hz, H_{b}), 6.57 (d, 2H, J=8.5 Hz, Hₐ), 3.99 (s, 4H, methylene), 3.90 (t, 2H, J=1.2 Hz, ArOCH₂), 3.47 (s, 8H, methylene), 2.60 (t, 2H, J=5.8 Hz, CH₂NMe₂), 2.47 (q, 2H, J=7.4 Hz, CH₃CH₂), 2.24 (s, 6H, N(CH₃)₂), 1.46 (s, 36H, ^{t}Bu), 0.92 (t, 3H, J=7.4 Hz, CH₃). ¹³CNMR (400 MHz, CD₂Cl₂): δ 170.72 (C29), 159.56 (C24), 157.27 (C4), 145.09 (C7), 142.54 (C18), 137.48 (C26), 132.21 (C20), 131.97 (C6), 129.98 (C19), 126.51-131.22 (C10-C15), 123.13 (C25), 120.84 (C21), 113.69 (C5), 93.32 (C23), 88.00 (C22), 81.21 (C30), 65.87 (C3), 60.04 (C27), 58.32 (C2), 57.32 (C28), 45.67 (C1), 29.37 (C16), 27.94 (C31), 13.54 (C17). C8 and C9 merged with CD₂Cl₂ peaks. The NMR assignments were assisted by ¹³C-¹H correlation (HET-CORR) 2-D spectra.

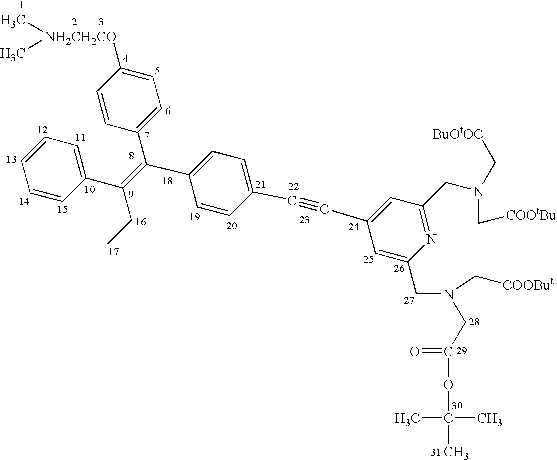

Flash column was performed under vacuum. The compound was initially loaded dry on the column after mixing with silica gel and then elution started. This time, before loading the compound on the column, the silica gel of the column was washed with 100 ml each of MeOH, CH₂Cl₂, EtOAC and hexane, kept under vacuum for about 1 hr to dry it completely and then the compound was loaded on it, and eluted each time with 50 ml of the solvent as before. Elution started with hexane→ethyl acetate→CH₂Cl₂→2% MeOH:CH₂Cl₂→4% MeOH:CH₂Cl₂→6% MeOH:CH₂Cl₂→6% MeOH:CH₂Cl₂ with 1 ml diisopropylamine as an additive, wherein the remaining compound came out. This time the fraction that came out in ethylacetate contained most of Compound 14 and was very pure as seen in the ¹H NMR spectrum. The fraction that came out in 6% MeOH:CH₂Cl₂ with 1 ml diisopropylamine as an additive also contained the remaining Compound 14 but was not as pure.

### 2.6 Synthesis of Compound 15

Compound 14 (0.4846 gm, 0.48 mmol) was dissolved in excess of trifluoroacetic acid (2.5 ml) and dichloromethane (2.5 ml) and was vigorously stirred in an ice-bath for 1.5 hrs. The trifluoroacetic acid was evaporated in vacuo without heating. The residue was triturated with ether (100 ml) and filtered to give Compound 15 (0.286 gm, 66%).

¹H NMR (250 MHz, MeOH-d₄): δ 7.61 (s, 2H, pyridyl), 7.56 (d, 2H, J=8.2 Hz, H_{d}), 7.28 (d, 2H, J=8.5 Hz, H_{c}), 7.10-7.20 (m, 5H, Hₑ), 6.83 (d, 2H, J=8.8 Hz, H_{b}) 6.68 (d, 2H, J=8.8 Hz, Hₐ), 4.31 (s, 4H, methylene), 4.19 (t, 2H, J=4.2 Hz, ArOCH₂), 3.73 (s, 8H, methylene), 3.51 (t, 2H, J=4.5 Hz, CH₂NHMe₂⁺), 2.93 (s, 6H, NHMe₂⁺), 2.47 (q, 2H, J=7.3 Hz, CH₃CH₂), 0.91 (t, 3H, J=7.3 Hz, CH₃).

### 2.7 Synthesis of Compound 16

The tetraacid Compound 15 (0.266 gm, 0.29 mmol) was dissolved in water (4 ml) and the pH was adjusted to 6.5 with solid NaHCO₃. Gd (III) chloride (0.090 gm, 1.659 mmol) in water (1 ml) was added over 15 min and the pH was maintained in the range of 5-7. After the mixture was stirred for 1.5 hrs at room temperature, the pH was raised to 8.5 with 1M NaOH and the precipitate was filtered off. Acetone was added to the filtrate to precipitate the Gd complex Compound 16 (0.056 gm, 20%). Compound 16 was filtered and washed with acetone. IR (KBr pellet): 3425, 2950, 1604, 1406 cm⁻¹ ν (OH, C≡C, C═O, CO).

### Example 3

### Binding of 17β-Estradiol- and Tamoxifen-Pyridine Conjugates and Metal Complexes Thereof to the Estrogen Receptor in Solution and in Cells

The assessment of the synthetic metalated estrogens and tamoxifen conjugates with regard to their binding affinity to the free ERα, is performed by competition between these molecules and tritiated estradiol for binding isolated recombinant ERα (Venkatesh et al., 2002). This procedure yields IC₅₀ displacement values for the inhibition of the binding of tritiated estradiol to the ER. Low IC₅₀ values correlates to high binding affinities. The reported IC₅₀ values of the steroidal metal complexes synthesized by Jackson et al. (2001) ranged between 39 to 5700 nM (the IC₅₀ of estradiol is 1 nM).

Another test involves measuring relative binding affinities of the metalated complexes of the conjugates for ERα in viable ER⁺ breast cancer cells using again a competitive radiometric binding assay (Venkatesh et al., 2002; Jackson et al., 2001). This assay also serves to characterize the ability of the probes to be transported into the cells and the nucleus, where most of the ER receptor resides. The results in whole cell assays obtained by Jackson et al. showed that cationic steroidal complexes described therein, exhibited similar receptor binding affinities compared to the neutral free ligand (Jackson et al., 2001). We thus assumed that proliferation of ER⁺ breast cancer cells in the presence of the conjugates and metal complexes thereof of the invention is a true indication that the compounds of the invention are transported to the cells, bind to estrogen and induce the same reaction sequence as estrogen.

We characterized the binding properties to ER-α of the Compounds 6, 7 and 16. The binding affinities were determined by performing a competitive radiometric-binding assay using tritiated estradiol and human recombinant ER. For comparison, the binding affinity of the antiestrogen, tamoxifen, was also measured. Table 1 presents the concentration of the competing ligand required to replace half of the ER bound tritiated estradiol, IC₅₀, and the calculated equilibrium concentration of the competing ligand that will bind to half of the ER binding sites, Ki.

TABLE 1
ERα competitive binding affinity of the 6, 7, 16 with
17β-estradiol

| Compound | IC₅₀, μM | Ki, μM |
|---|---|---|
| Compound 6 | 4.4 | 0.40 |
| Compound 7 | 8.6 | 0.78 |
| Compound 16 | 5.3 | 0.48 |
| Tamoxifen | 0.05 | 0.005 |

### Example 4

### Compounds 6 and 7 Exhibit Estrogenic Activity In Vitro

The agonistic or antagonistic effects of the conjugates and metal complexes thereof were studied by their capacity to enhance or arrest cell growth, respectively. It was assumed that tamoxifen metal complexes will act as antagonists whereas the estrogen metal complexes act as agonists.

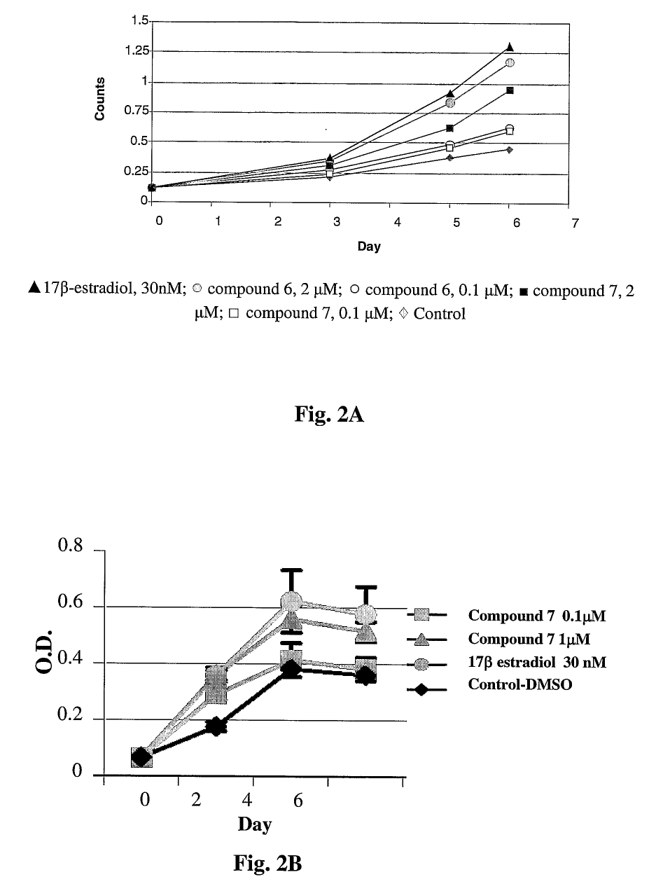

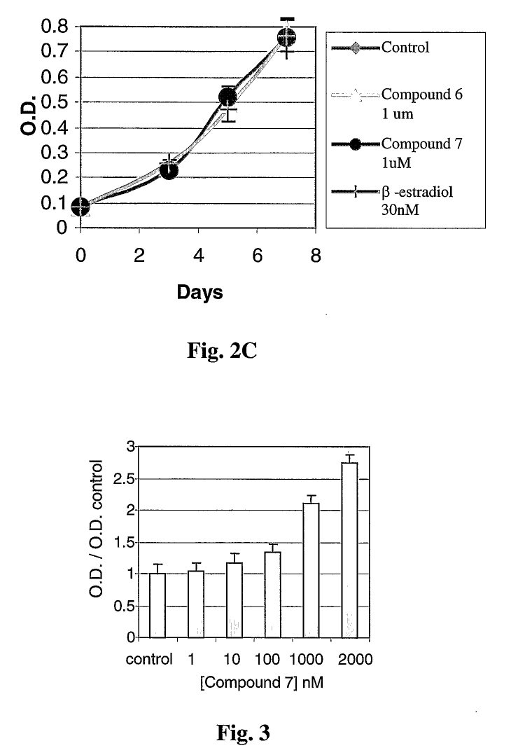

In order to test the ability of the conjugates and metal complexes to enter the cells and stimulate their growth in the same manner as free 17β-estradiol, the time and dose dependent induction of cell proliferation by the 17β-estradiol-17α-ethynyl-pyridine conjugate 6 and its Gd complex 7 were studied in three different estrogen receptor positive (ER⁺) human breast cancer cell lines MCF7, T47D and ZR-75-1. The cells were cultivated in phenol red-free medium and estrogen-free DMEM and then treated for several days with 30 nM 17β-estradiol, 30 nM or 1 μM of compound 6 or 7. The number of cells was determined spectrophotometrically using the MTT [3-(4,5-dimethylthiazol-2-yl)-2,5-diphenyltetrazolium bromide] method. The samples are read using an ELISA plate reader at a wavelength of 570 nm. The amount of color produced is directly proportional to the number of viable cells. The proliferation curves of ER⁺ T47D cells and MCF7 cells are shown in FIG. 2A and FIG. 2B, respectively

For all three cell-lines, we found that both the Gd complex and the metal free conjugates stimulated the growth of the cells in a similar manner to that of estrogen. For a full stimulating effect, the dose of the Gd-complex 7 was raised to 1 μM. The metal-free conjugate 6 was toxic at high concentrations, as is usually the case with high estradiol concentrations. The dose of the metalated conjugate 7 was very low in physiological/pharmacological terms.

To verify their specificity to ER⁺ cells, we measured the time and dose dependent effects of compounds 6 and 7 on the proliferation of the estrogen-receptor negative (ER⁻) human breast cancer cell line, MDA-MB-231. As shown in FIG. 2C, the proliferation of these cells was neither sensitive to 17β-estradiol nor to the unmetalated conjugate 6 or the Gd Complex 7.

The dose response of compound 7 on the proliferation of T47D human breast cancer cells is shown in FIG. 3. The cells were cultured for 6 days in estrogen-free medium containing various concentrations of compound 7. The vehicle (water) was added as control and served to normalize the changes in cell number. The results clearly demonstrate a dose-dependency for the ligand. Compound 7 induced cell proliferation as 17β-estradiol, but at different doses. The dose of the metalated complex 7 needed for induction of cell proliferation, in comparison with the estrogen 17β-estradiol, indicate high affinity of 7 to ERα. Although somewhat high, the optimal dose was still in the pharmacological range of micromolar.

### Example 5

### The effect of Compound 7 on T₁ Relaxation Rate of Water

The T₁ and T₂ relaxivity of the MRI probes, i.e., the Gd complexes of the invention, were measured in saline solutions using standard protocols (i.e inversion recovery for T₁ relaxivity and Carr-Purcell-Meiboom-Gill (CPMG) for T₂ relaxivity).

The capacity of the Gd complex 7 to serve as a relaxation contrast agent was determined by measuring its relaxivity. The T₁ and T₂ relaxation rate (R₁ and R₂) of water in the presence of increased concentrations of compound 7 were measured at 4.7 Tesla (Bruker, Biospec 4.7 T/30 cm bore). For the T₁ relaxivity measurement, a spin echo sequence was employed with a varying repetition time-TR (11 TRs, from 50 to 15,000 msec) and a fixed echo time of 23 msec. The T₁ relaxivity, defined as: R1_{c}-R1_{c=0}/[compound 7] thus measured in phosphate buffer saline solution, was 8 mM/sec. The T₂ relaxivity measured was 30 mM/sec at 25° C.

T₁ relaxivity depended to some extent on the instrument (field strength) and the nature of the solution (water, saline etc.) in which the Gd-complex was dissolved. The accuracy of measurement may depend on the method used to measure the change in T₁ for a given concentration of Gd-complex 7. Therefore, inaccuracy in the measurement data also depends on the accuracy of the concentration of the contrast agent. A combination of all these inaccuracies resulted in somewhat different T₁ relaxivity values obtained for different measurements, but all values were close to 8 nm/sec.

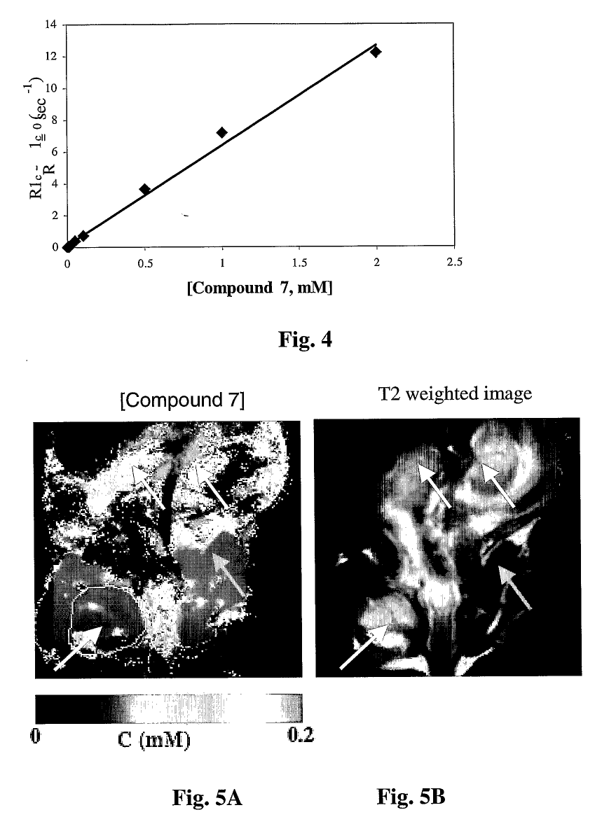

FIG. 4 shows a plot of the change in the water T₁ relaxation rate in the presence of increased concentrations of the Gd complex 7. The T₁ relaxivity was determined from the slope.

We determined the MRI properties of the novel paramagnetic ER ligands. T1 and T2 relaxation rates of the water protons were measured as a function of the concentration of the Gd complex 7. The T1 and T2 relaxivities (changes in T1 and T2 relaxation rates per a unit concentration of 7) measured at 4.7 Tesla were found to be 7.99±0.05 mM⁻¹ sec⁻¹ and 30.8±1.0 mM⁻¹ sec⁻ respectively. These values were significantly higher than the T1 and T2 relaxivities found for the gadolinium chelate of 2,2′,2″,2′″-[(pyridine-2,6-diyl)bis(methylenenitrilo) tetrakis(acetic acid) of 6.0±0.2 mM⁻¹ sec⁻¹ and 6.6±0.8 mM⁻¹ sec⁻¹, respectively, and those reported for gadolinium diethylenetriamine pentaacetic acid (Gd DTPA) (4.2 mM⁻¹ sec⁻¹ for both T1 and T2 relaxivities). Thus, the high relaxivities of the Gd complex 7 is in favor of its utilization as a contrast agent.

### Example 6

### Non-Invasive In Vivo MRI Monitoring of the Binding and Distribution of the Gd Complex 7

Animals: CD1-NU immunodeficient female mice (6-10 weeks old, 20-25 g weight) were provided by the Animal Unit of the Weizmann Institute of Science. During the MRI experiments, the animals were anesthetized by inhalation of 1% Isoflurane in an O₂:N₂O (3:7) mixture, applied through a nose cone. All animals were handled according to the regulations formulated by the Institutional Animal Care and Use Committee (IACUC) of the Weizmann Institute of Science (Rehovot, Israel).

Toxicology: The probes (e.g. Gd complex 7) were injected into the tail vein of CD1-NU mice at a varying dose in the range of 0.024 to 1.0 mmol/kg weight.

Tumors: ER⁺ human breast cancer MCF-7 cells were implanted orthotopically into the mammary gland of the CD1-NU immunodeficient female mice, as previously described (Paran et al., 2004). The mice were ovariectomized to eliminate any exogenous estrogen. Initially a pellet of 17β-estradiol (Innovative Research, Florida) was implanted before the cells were injected, to ensure growth of the tumors. Within 3 weeks, tumors developed to a size of about ˜0.5 ml and consisted of highly proliferating cells. At this stage we removed the estrogen pellet and after a week administered the MRI probe. The extent of accumulation in the tumors was studied in vivo and in excised tumors and was then compared to results obtained by the standard immunostaining technique.

MRI: The accumulation of the MRI probe was tested by scanning the tumors before the injection of the probe and at times after the injection, using T₁ weighted and T₂ weighted pulse sequences. MR images were recorded with a 4.7 Tesla scanner (Bruker; Biospec 4.7 T/30 cm bore, dedicated Doty resonator, 3 cm diameter, 200 MHz for protons). The MRI parameters for scanning T₂-weighted images were: 2D Spin echo with: TE/TR=69 msec/4000 msec, field of view (FOV) 5 cm, slice thickness 1.2 mm and 128×256 matrix. For T₁ measurements two pulse sequences were employed: 1. Inversion recovery pulse sequence with varying T₁ (n=20) ranging from 10 to 10000 msec, TE/TR=3.15 msec/15 msec, flip angle 10 degrees; 2. Fast gradient echo sequence. The spatial resolution was as in the T₂ weighted image except the matrix was 128×128.

Distribution of the MR probes: The distribution of the Gd complex 7 in the blood, inner organs and the tumors was monitored over a period of 24 h after a bolus injection of 7 into the tail vein at a dose ranging from 0.024 to 0.4 mmol/kg.

In the present example, we employed the unique ability of MRI to monitor non-invasively specific enhancement due to binding of the ER-ligand-Gd complex to the estrogen receptor in ER⁺ MCF7 tumors. We first found that Gd-complex 7 is not lethal and does not cause obvious adverse effects up to a high dose of 0.4 mmol/kg weight, injected intravenously to CD1-NU mice. Repetitive administration, 3-7 days apart, appeared to be harmless as well.

The presence of paramagnetic probes in cells or tumors is expected to affect T₁ and T₂ nuclear relaxation rates, as well as to evoke a susceptibility T₂* effect. We have estimated that in MCF7 tumors, the expected changes due to the presence of an MRI probe bound to ER would be about 5-10% in T₁ and 10-17% in the susceptibility effect (T₂*).

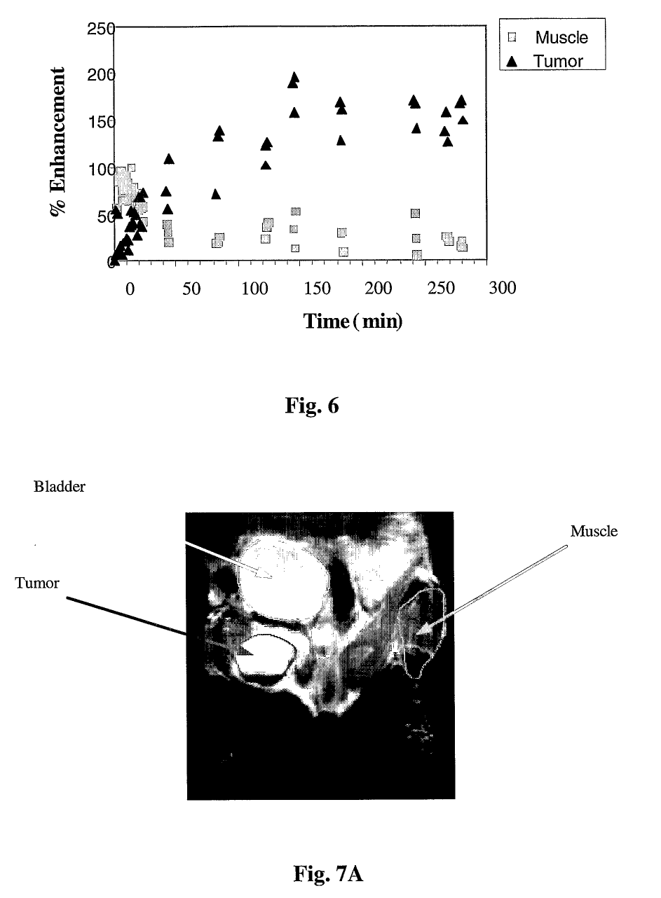

We monitored the distribution of the Gd complex 7 throughout the body of the CD1-NU mice implanted orthotopically with ER⁺ MCF7 breast tumors. Sequential images of the tumor and other parts of the body, including the kidneys and the bladder, were recorded before and after administering 7 (FIGS. 5 and 6).

The distribution of 7 was compared to that of GdDTPA (the contrast agent currently in clinical use) in the same mice using a bolus injection of the same dose (0.4 mmol/kg). Compound 7 was found to enter and clear the intracellular compartment slowly compared to GdDPTA, which cannot enter the cells. The clearance of 7 through the kidneys into the urine was several folds slower than that of GdDTPA and full clearance occurred only 24 hours after administration of 7 (FIG. 6).

FIGS. 5A-5B show T₂-weighted MR image (5B) and map of apparent concentration of the Gd complex 7 (5A) in a body slice of a CD1-NU mouse with orthotopic MCF7 breast tumor.

The contrast agent distribution was monitored, alternating between two pulse sequences: 1. T₁-weighted, 3D gradient echo with TE/TR of 4.3/18.3 ms; a 30° flip angle, 2. 2D inversion or saturation recovery sequence with varying T₁ (n=20) ranging from 10 to 10000 msec, and gradient echo acquisition with TE/TR of 3.5/15 msec and 100 flip angel. The latter sequence enabled us to map the T₁ relaxation time. The spatial resolution was the same for both sequences and during the whole experiment time, namely, 0.2×0.4×1.2 mm³. Susceptibility gradient echo and T₂ weighted spin echo protocols were employed to generate the MR images of the animal.

The concentration map was obtained from T₁ relaxation measurements before and 2 h after terminating slow infusion of the Gd complex 7. The infusion dose was high, 0.4 mmol/kg, and lasted for 1 hour. The final concentration in the tumor and muscle was similar, however, in some other parts including the kidney, the concentration was higher. In fat tissue and other internal regions we did not observe accumulation of the ligand either due to its absence or failure of the T₁ fitting.

### Example 7

### Non-Invasive In Vivo MRI Monitoring of the Binding of the Gd Complex 7

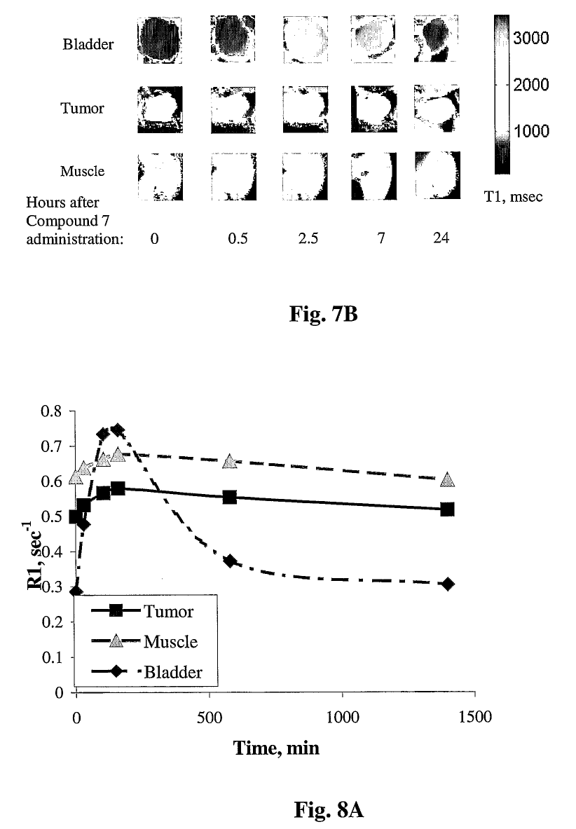

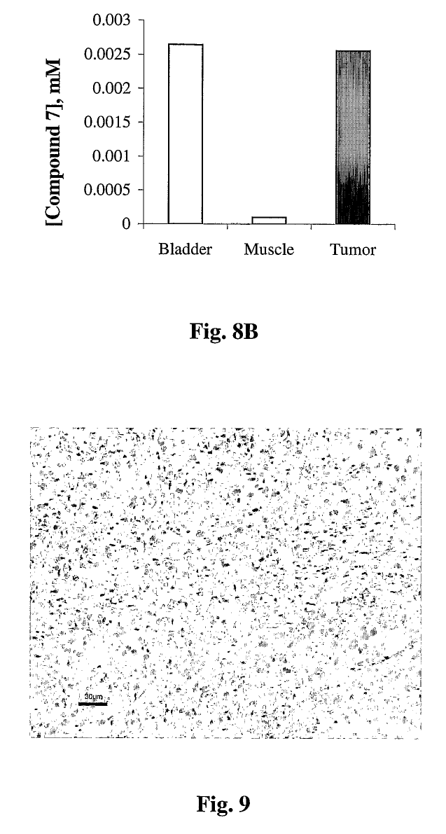

Several protocols of administration of the Gd complex 7 were examined: a high and low dose bolus injection of 0.4 mmol/kg and 0.024 mmol/kg, respectively (FIGS. 7 and 8), and a slow infusion (drip) protocol over 60 min of the high dose.

FIG. 6 shows MRI signal enhancement (%) after a bolus administration of 0.4 mmol/kg of the Gd complex 7 in the MCF7 breast tumor and muscle tissue of the mouse. The percent (%) enhancement values, defined as the signal intensity at time t [I(t)] minus the pre-contrast intensity [I(0)] divided by I(0) times 100: {[I(t)−I(0)]/I(0)}×100, were obtained from T₁-weighted images. As shown in FIG. 6, the enhancement in the tumor, due to the high dose (0.4 mmol/kg) administration of 7 persisted throughout the experimental time (about 5 hours) whereas that of the muscle declined close to pre-contrast administration, suggesting trapping and binding of the Gd complex 7 in the tumor. The MRI was performed on the same scanner as in FIG. 5 above, using fast 3D gradient echo: TE/TR=4.3/18.3 msec, FOV=5×5×2.16 cm³ matrix: 128×256×16, flip angle 30 degrees.

To determine the ER-binding of 7 administered at low concentration (0.024 mmol/kg), we performed measurements up to 24 after its administration, when all the tissues appeared to be cleared from the probe. FIGS. 7A-7B show the time course of the T₁ relaxation in the bladder, orthotopic MCF7 breast tumor and muscle of a female CD1-NU immunodeficient mouse after a bolus administration of a low dose of 7 (0.024 mmol/kg) (7B) and the area of each organ as marked on the upper T₂-weighted image (FIG. 7A). The images at 0, 0.5 and 2.5 h were recorded with the anaesthetized mouse in the same position. After the 2.5 h measurement, the mouse was returned to its cage and was again scanned under anesthesia at the 7 and 24 hours time points. Hence, the localization was similar but not identical to that of the earlier time points. The central slice of each organ is presented. As shown in FIG. 7B, dramatic changes in the bladder relative to those in the muscle and tumor are observed. The MRI scanner and measurement parameters were similar to those described above for FIG. 5, except for the matrix: 256×256 and the slice thickness: 1.5 mm.

The binding of compound 7 to ER was further assessed by the T₁ relaxation rate of water in the tumor, R₁, obtained from the measurements performed 24 hours after Gd-complex 7 administration. We found that the ER⁺ breast tumors still exhibited small but persistent (n=3) increased tumor T₁ relaxation rate (R₁), while in the muscle this rate returned to that measured before administrating the ER-Ligand-Gd 7 (FIGS. 8A-8B). This small increase suggested binding of the ER-ligand-Gd 7 to the ER in the tumor. Immunohistochemical staining of the tumor for ERα confirmed the high abundance of this receptor predominantly in the nuclei (FIG. 9).

FIG. 8A shows changes with time in T₁ relaxation rate, R₁, in orthotopic MCF7 breast tumor, muscle tissue and bladder, after bolus administration of ER-Ligand-Gd 7 (0.024 mmol/kg) into the tail vein of a female immunodeficient mouse. FIG. 8B shows change in apparent concentration (calculated from the measured relaxation rates) 24 hours after administration of 7. The T₁ measurements were performed as described in FIG. 7. The T₁ values present average values over the whole tumor volume, bladder volume and region of interest (ROI) of muscle (demonstrated in FIG. 7). It is to be noted that no residual amount was left in the muscle but the tumor still exhibited presence of the ER-Ligand-Gd 7 (2.5 μM). The low concentration in the bladder reflected the final tracer amounts that reached this organ from the whole body. In two other experiments the same difference between tumor and muscle concentration was obtained. In one experiment the bladder concentration was also low compared to the tumor. FIG. 6B shows that no residual amount of 7 was left in the muscle, but the tumor still exhibited presence of the probe (2.5 μM). The low concentration in the bladder reflected the final tracer amounts that reached this organ from the whole body. The concentrations were calculated from the measured relaxation rates.

Immunohistochemical staining of the tumor for ERα confirmed the high abundance of this receptor predominantly in the nuclei (FIG. 9). The staining was performed as previously described (Bevitt et al., 1997) using the monoclonal antibody NCL-ER-6F11/2 (Novocastra Laboratories, Newcastle upon Tile, UK). As shown in FIG. 9, a large fraction of the nuclei was stained by the antibody. However, not all nuclei exhibited this staining. Since the tumors were grown in the presence of slow release of 17β-estradiol, we predict that the ER level was down regulated.

### Example 8

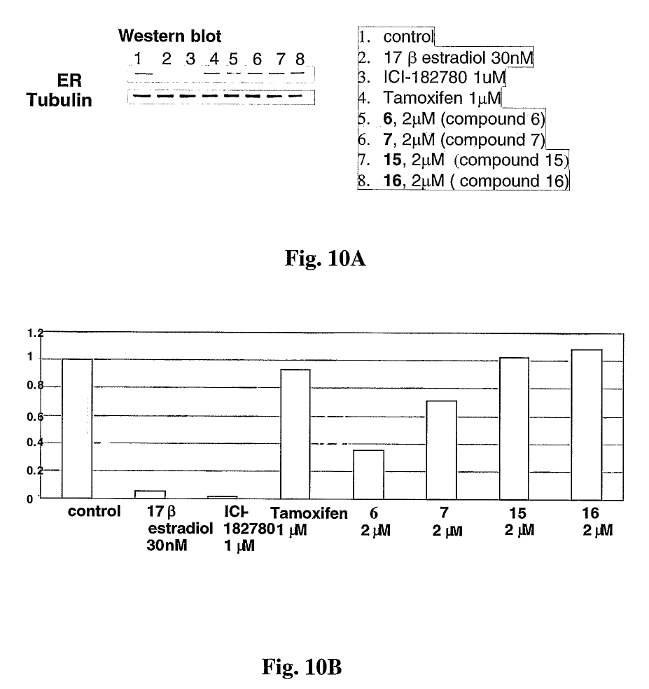

In an additional study, we characterized the agonistic or antagonistic effects of the new ligands and complexes in terms of the proliferation of breast cancer cells (MCF7, T47D ER positive cells), and the effect on ERα degradation. We found that the estradiol derivatives Compound 6 and corresponding Gd complex 7 are agonistic and at 2 μM induce cell in a similar manner to estradiol (E2). The tamoxifen derivatives Compound 15 and corresponding Gd complex 16 did not elicit an estrogenic effect on the proliferation as the tamoxifen moiety is antagonistic. We also found that 6 and 7 induced partial degradation of the receptor, whereas 15 and 16 acted like tamoxifen, as shown in FIG. 10A.

We then carried out a Western blot for determining the level of the estrogen receptor protein using an anti-ER antibody to identify ER in the blot. We referenced the level of ER protein on the blot to another protein, tubulin, which remains constant and is not affected by the estrogen or by the ER-ligands of the invention. FIG. 10A depicts the Western blot and FIG. 10B depicts quantitation of the blot: the amount of ER relative to tubulin, assuming tubulin is constant under all treatment manipulations.

### Example 9

### In Vivo Tests of f Gd Complex 7

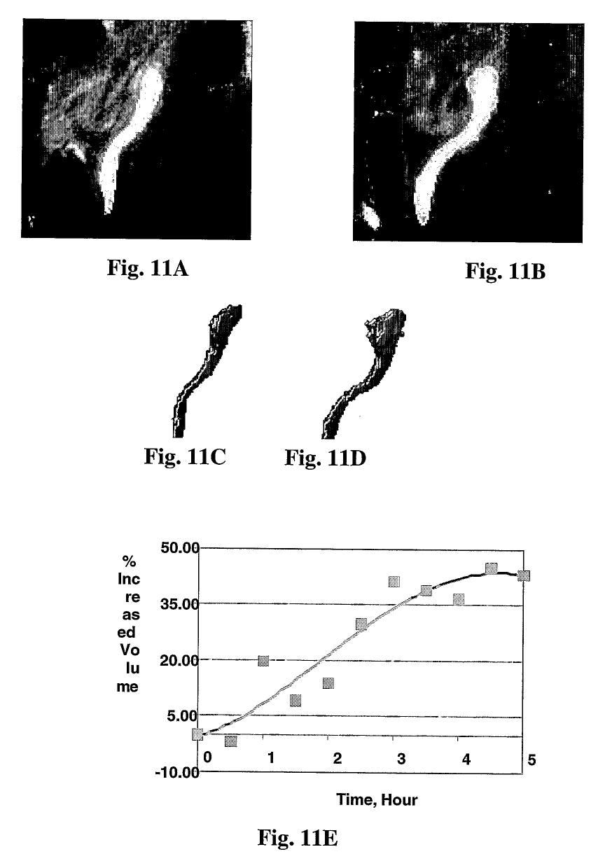

We developed and applied in vivo tests of the Gd complex 7 in the classical target organ of estrogen (the rat uterus). We found that the uterus of the ovariectomized rat is suitable for the MRI studies of the interaction of 7 to ER. In order to be able to delineate the uterus, we applied a newly developed segmentation algorithm that enables us to automatically monitor changes in the entire uterine volume and in the changing nuclear relaxation rates due to interaction with 7. We showed that injection of 7 (at dose of 0.024 mmol/kg) to ovariectomized rates elicited an agonistic effect in the uterus demonstrated by water imbibition and increased uterine volume. The endometrium volume increased by 45%, over the entire monitoring period of 5.5 hours, indicating response to the Gd complex 7, as shown in FIGS. 11A-11E. Parallel measurements of signal enhancement due to increased T1 relaxation rate in the presence of 7 in the uterus and in the muscle tissue revealed differences indicating specific binding of 7 in the uterus.

The endometrial volume in ovariectomized female rats after a bolus administration of 7 (0.024 mmol/kg) is presented in FIGS. 11A-11E. FIG. 11A depicts the T2-weighted images prior to administration and FIG. 11B depicts the T2-weighted images after 5 hours of administration of compound 7 (the endometrium is circled in green) with the corresponding 3D automatically delineated right horn (FIGS. 11C and 11D) and the corresponding changes in the volume during the entire time course (FIG. 11E).

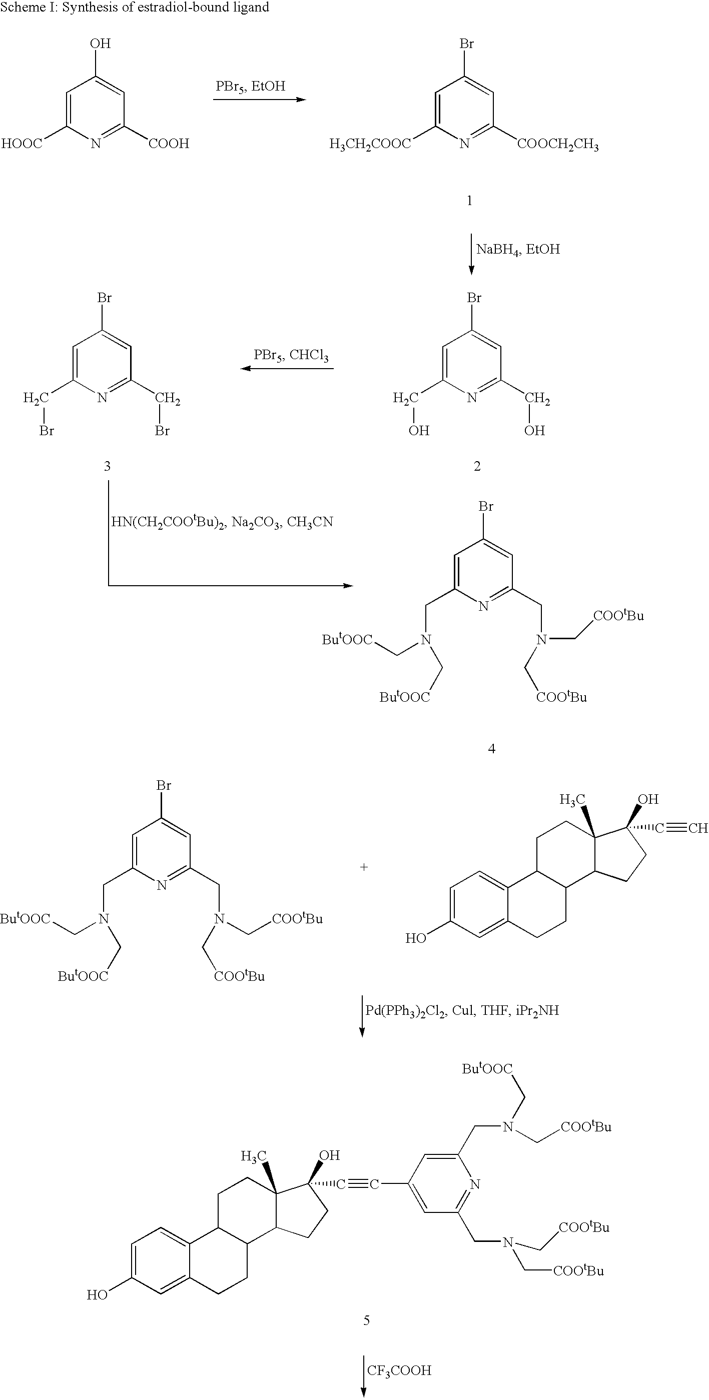

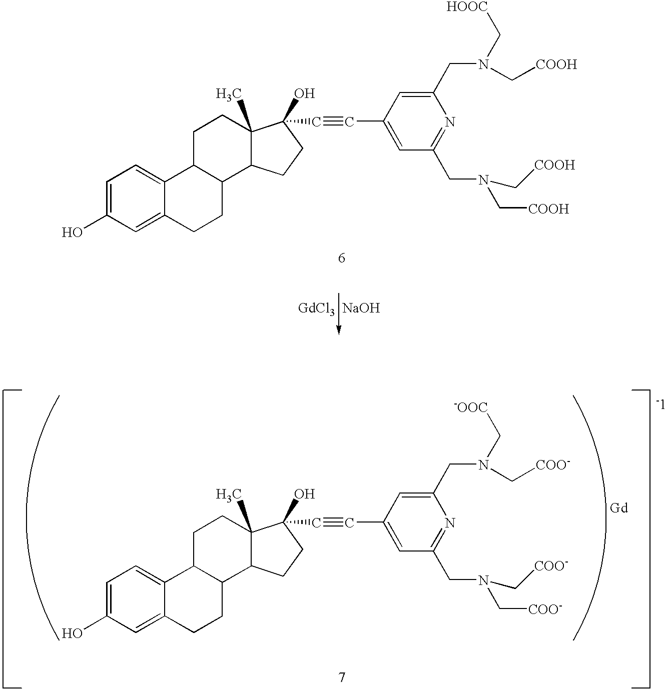

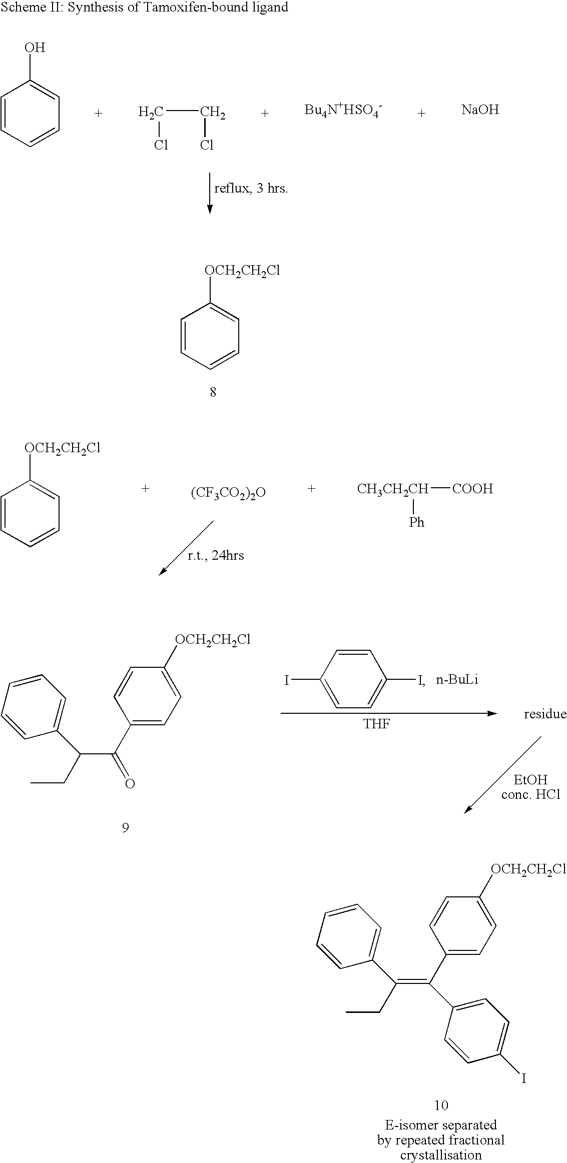

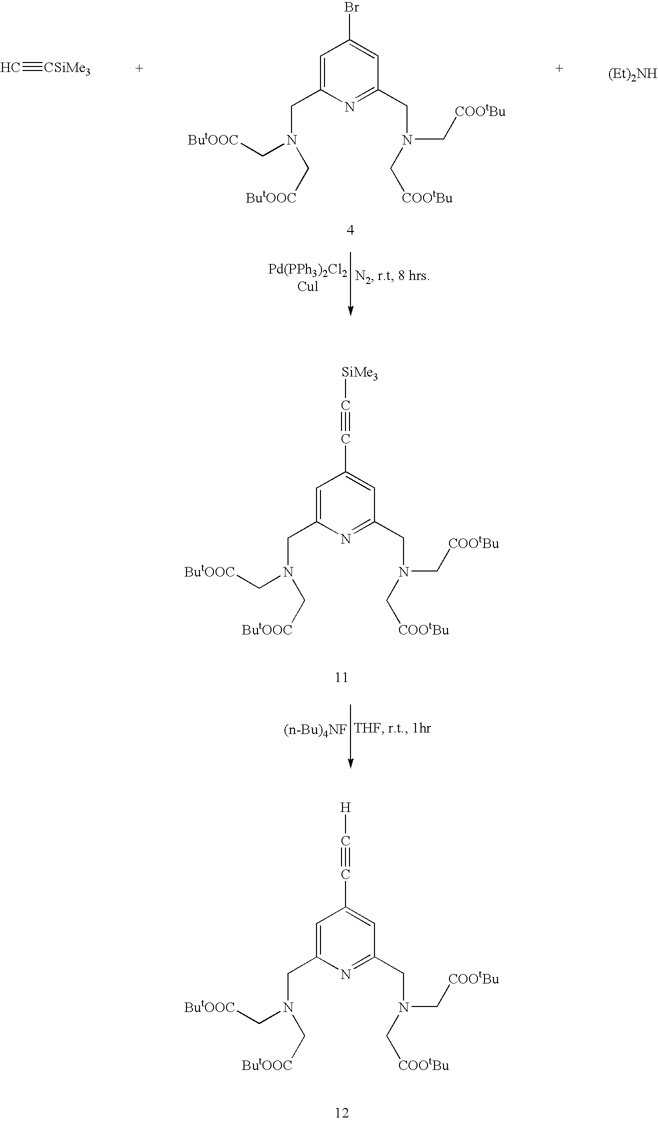

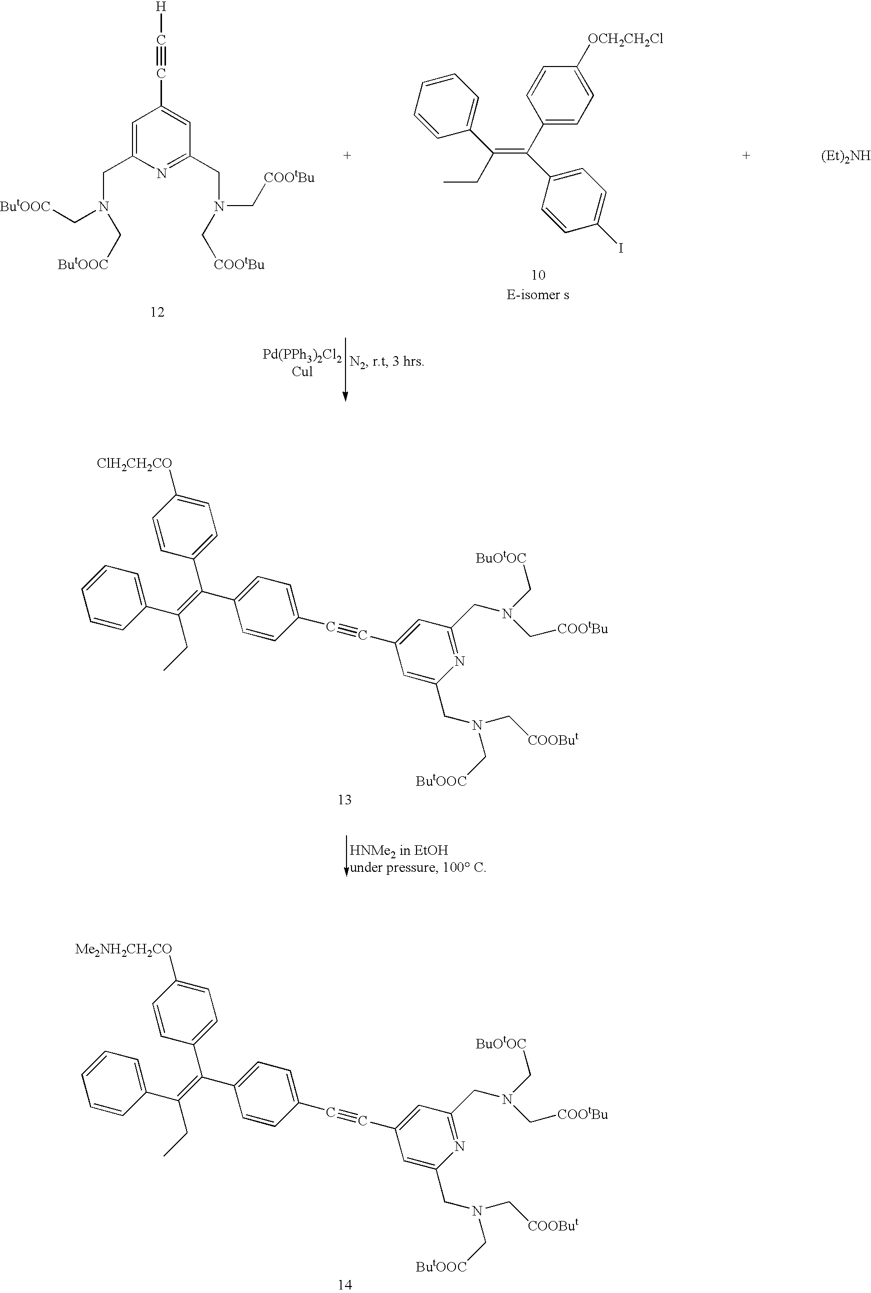

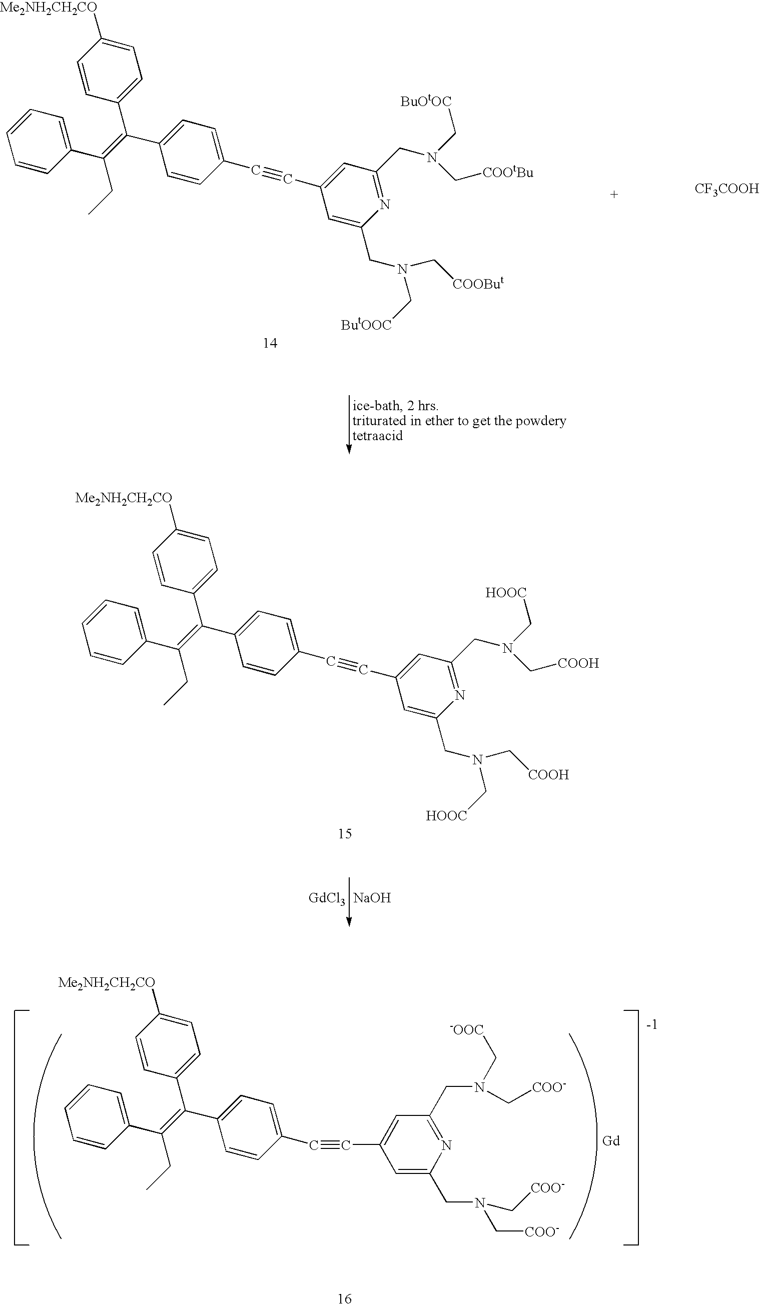

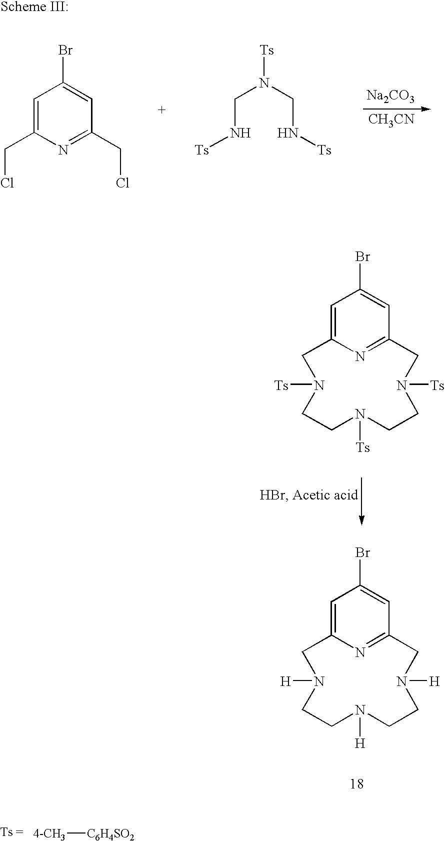

### REFERENCES

Barnes D M, R R Millis, LVAM Beex, S M Thorpe and R E Leake, “Increased use of immunohistochemistry for estrogen receptor measurement in mammary carcinoma: the need for quality assurance”, European J. of Cancer, 34:1677-1682, 1998.
Bevitt D J, I D Milton, N Piggot, L Henry et al., “New monoclonal antibodies to estrogen and progesterone receptors effective for paraffin section immunohistochemistry”, J. Pathol., 183:228-32, 1997.
Boxerman J L, L M Hamberg, B R Rosen and R M Weisskoff, “MR contrast due to intravascular magnetic susceptibility perturbations”, Magn. Reson. Med., 34(4):555-66, 1995.
Curtet C, F Maton, T Havet, M Slinkin et al., “Polylysine-Gd-DOTAn coupled to anti-CEA F(ab′)2 fragments as potential immunocontrast agents. Relaxometry, biodistribution, and magnetic resonance imaging in nude mice grafted with human colorectal carcinoma” Investigations in Radiology, 33:752-761, 1998.
Dennie J, J B Mandeville, J L Boxerman, S D Packard, B R Rosen and R M Weisskoff, “NMR imaging of changes in vascular morphology due to tumor angiogenesis”, Magn. Reson. Med., 40(6):793-9, 1998.
Fisher B, J P Constantino, J D L Wickerham et al., “Tamoxifen for prevention of breast cancer: report of the National Surgical Adjuvant Breast and Bowel Project P-1 Study”, J. Natl. Cancer Inst., 90:1371-88, 1998.
Furman E, E Rushkin, RMargalit, P Bendel and H Degani, “Tamoxifen induced changes in MCF7 human breast cancer: in vitro and in vivo studies using nuclear magnetic resonance spectroscopy and imaging”, J. Steroid. Biochem. Mol. Biol., 43:189-195, 1992.
Fuqua S A, R Schiff, I Parra et al., “Expression of wild type estrogen receptor beta and variant isoforms in human breast cancer”, Cancer Res., 59: 5425-5428, 1999.
Grohmann A and F Knoch, Inorganic Chemistry, 35: 7932, 1996.
Hansen M B, S E Nielsen and K Berg, “Re-examination and further development of a precise and rapid dye method for measuring cell growth/cell kill”, J. Immunol. Methods, 119:203-210, 1989.
Hardcastle I R, M G Rowlands, J Houghton, I B Parr, G A Potter and M Jarman, J. of Med. Chem., 38:241-248, 1995.
Harvey J M, G M Clark, C K Osborne and D C Allred, “Estrogen receptor status by immunohistochemistry is superior to the ligand-binding assay for predicting response to adjuvant endocrine therapy in breast cancer”, J. of Clinical Oncology, 17:1474-1481, 1999.
Hunter D H, L G Luyt, “Single isomer technetium-99m tamoxifen conjugates” Bioconjugate Chemistry, 11: 175-181, 2000
Jackson A, J davis, R J Pither, A Rodger and M J Hannon, “Estrogen-derived steroidal metal complexes: Agents for cellular delivery of metal centers to estrogen receptor-positive cells”, Inorg. Chem., 40:3964-3973, 2001.
Jakesz R, C A Smith, S Sitken, K Huff et al, “Influence of cell proliferation and cell cycle phase on expression of estrogen receptor in MCF7 breast cancer cells”, Cancer Res., 44:619-625, 1984.
Katzenellenbogen J A, R E Coleman, R A Hawkins et al., “Tumor receptor imaging: proceeding s of the national cancer institute workshop, review of current work, and prospective for further investigations”, Clinical Cancer Research, 1:921-932, 1995.
Kenna R P, J Zhong and J C Gore, “Intravascular susceptibility contrast mechanisms in tissues”, Magn. Reson. Med., 31(1):9-21, 1994.
Khan S A, K A Yee, C Kaplan and J F Siddiqui, “Estrogen receptor a expression in normal human breast epithelium is consistent over time”, Int. J. Cancer, 102:334-337, 2002.
Kuiper G G J M, E Enmark, M Pelto-Huikko et al, “Cloning of a novel estrogen recepto expressed in rat prostate and ovary”, Proc. Natl. Sci. OSA, 93: 5925-30, 1996.
Leclercq G, “Molecular forms of estrogen receptor in breast cancer”, J. Steroid. Biochem. Mol. Biol., 80:259-272, 2002.
Leygue E, H Dotzlaw, P H Watson and L C Murphy, “Altered estrogen receptor alpha and beta messenger RNA expression during breast tumorogenesis, Cancer Res., 58: 3197-201, 1998.
Li C I, J R Daling and I E Malone, “Incidence of invasive breast cancer by hormone receptor status from 1992 to 1998”, J. Clin. Oncol. 21:28-34, 2003.
Macgregor J I, V C Jordan, “Basic guide to the mechanism of antiestrogen action”, Pharm. Reviews, 50:151-96, 1998.
McGuire W L, “Hormone receptors: Their role in predicting prognosis and response to endocrine therapy” Semin. Oncol., 5: 428-33, 1978.
Mortimer J E, Dehadashti F, Siegel B A, Katenellenbogen et al.”, Positron emission tomography with 2-[18F]fluoro-2-deoxy-D-glucose and 16α-[18F]fluoro-17β-estradiol in breast cancer: correlation with estrogen receptor status and response to systemic therapy”, Clinical Cancer Research, 2:933-939, 1996.
Mull E S, V J Sattigeri, A L Rodriguez and J A Katzenellenbogen, “Aryl cyclopentadienyl tricarbonyl rhenium complexes: Novel ligands for the estrogen receptor with potential use as estrogen radiopharmaceuticals”, Bioorg. & Medic. Chem., 10:1381-98, 2002.
Muramatsu M and S Inoue, “Estrogen receptors: How do they control reproductive and non reproductive functions?” Biochem. Biophys. Res. Commun., 270:1-10, 2000.
Osborne C K, R M Elledge and A W Fuqua, “Estrogen receptors in breast cancer therapy”, Scien. Am. Science & Med., 32:41, 1996.
Paran Y, P Bendel, RMargalit and H Degani, “Water diffusion in the different microenvironments of breast cancer”, NMR Biomed. 17(4):170-80, 2004.
Rijks L J M, P J M Bakker, Van G Tienhoven, L A Noorduyn, G J Boer, R C Rietbroek, C W Taat, A G M Janssen, C H N Veenhof and E A van Royen, “Imaging of estrogen receptors in primary and metastatic breast cancer patients with Iodine-123-labeled Z-MIVE”, J. of Clinical Oncology 15(7):2536-2545, 1997.
Ronen S and H Degani, “Studies of the metabolism of human breast cancer spheroids by NMR”, Magn. Reson. Med. 12:274-281, 1989.
Saji S, E V Jensen, S Nilsson et al., “Estrogen receptors alpha and beta in the rodent mammary gland”, Proc. Natl. Acad. Sci. USA 97: 337-42, 2000.
Saji S, J M Knowlden, J M W Gee et al., “A possible divergent role for the estrogen receptor alpha and beta subtypes in clinical breast cancer”, Int. J. cancer, 89: 2029-212, 2000.
Shyamala G, Y C Chou, S G Louie et al., “Cellelar expression of estrogen and progesterone in mammary glands: regulation by hormones, development and aging”, J. Sterod. Biochem. Mol. Biol., 80: 137-148, 2002.
Takalo H and J Kankare, “Synthesis of dimethyl and diethyl 4-(phenylethylyl)-2,6-pyridinedicarboxylate”, Acta Chemic. Scandin., 41:219-221, 1987.
Takalo H, P Pasanen and J Kankare, “Synthesis of 4-(phenylethynyl)-2,6-bis[N,N-bis-(carboxymethyl)aminomethyl]pyridine”, Acta Chemic. Scandin., 42:373-377, 1988.
Venkatesh N, Yzaltsman, D Somjen, B Gayer et al, “A synthetic peptide with estrogen-like activity derived from a phage-display peptide library”, Peptides, 23:573-80, 2002.
Vera D R, M H Buonocore, E R Wisner, R W Katzberg and R C Stadalnik, “A molecular receptor binding agent for magnetic resonance imaging of the liver”, Academic Radiology, 2:497-506, 1995.
Weisskoff R M, C S Zuo, J L Boxerman and B R Rosen, “Microscopic susceptibility variation and transverse relaxation: theory and experiment”, Magn. Reson. Med., 31(6):601-10, 1994.

1. A conjugate of the general formula I, II or III: 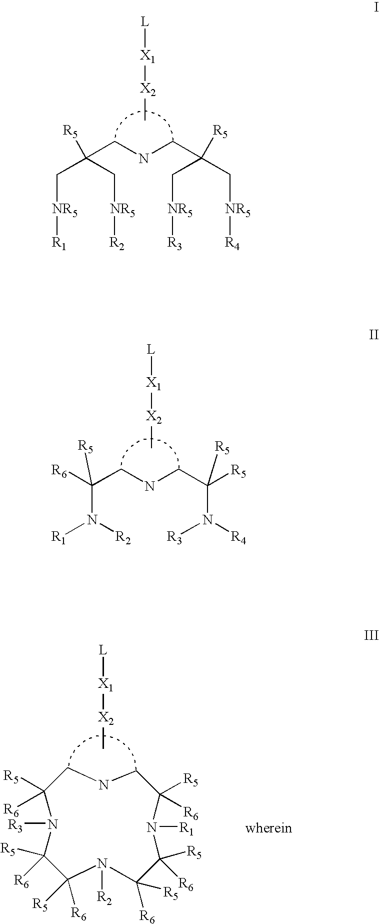
L is a moiety of a ligand of a receptor associated with malignant tumors or a chemotherapeutic drug moiety;
X₁ is a C₂-C₁₀ hydrocarbylene chain;
X₂ is phenylene or a covalent bond;
R₁ to R₄ in the conjugates of formulas I and II and R₁ to R₃ in the conjugate of formula III, each is H, (C₁-C₄) alkyl or CH₂R₇;
R₅ and R₆ each is H or (C₁-C₄) alkyl;
R₇ is selected from the group consisting of —COOR₈, —COO⁻, 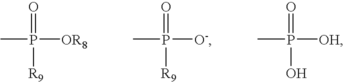
—PO₃²⁻ and —CONHR₈;
R₈ and R₉ each is selected from the group consisting of H, (C₁-C₄) alkyl, phenyl and benzyl, wherein the phenyl or benzyl can be substituted by at least one group selected from the group consisting of halogen, (C₁-C₄) alkyl and OR₅;
the dotted lines, when present, define that the N atom and the 2 adjacent C atoms are part of a mono- or polycyclic ring;
and metalated complexes of a conjugate of formula I, II or III, wherein R₁ to R₄ each is —CH₂R₇ and R₇ is —COOR₈, —COO⁻, PO₂(R₈)(R₉), PO₃²⁻, PO₃H₂ or —P(R₉)O₂⁻ with a paramagnetic lanthanide metal selected from the group consisting of Gd (III), Eu(III), Dy(III), Tb(III), Tm(III), Yb(III) and Pr(III), and of a conjugate of formula I, II or III, wherein R, to R₄ each is H, (C₁-C₄) alkyl or —CH₂R₇ and R₇ is as defined above, with a paramagnetic transition metal selected from the group consisting of Mn(II), Co(III), Ni(III), Fe(II) and Fe(III).

2. A conjugate according to claim 1, wherein L is a moiety of a ligand of a steroid receptor associated with malignant tumors.

3. A conjugate according to claim 2, wherein said steroid receptor is an estrogen receptor and said ligand is selected from the group consisting of 17β-estradiol, estrone, estriol, tamoxifen and analogs of the foregoing.

4. A conjugate according to claim 2, wherein said steroid receptor is the progesterone receptor and said ligand is a progestin such as progesterone.

5. A conjugate according to claim 2, wherein said steroid receptor is the androgen receptor and said ligand is testosterone.

6. A conjugate according to claim 1, wherein L is a moiety of a ligand of a non-steroidal hormone receptor associated with malignant tumors or other abnormalities such as luteinizing hormone/human chorionic gonadotropin receptors, human growth hormone receptor, and somatostatin receptor, or L is a ligand of another receptor such as a retinoic acid receptor.

7. A conjugate according to claim 1, wherein L is a moiety of a chemotherapeutic drug.

8. A conjugate according to claim 1, wherein X₁ is a saturated or unsaturated aliphatic C₂-C₁₀ hydrocarbylene chain optionally interrupted by at least one atom or radical selected from the group consisting of —O—, —S—, —N(R₅)—, —CO—N(R₅)—, —N(R₅)—CO—, —COO—, —OOC—, —N═N—, —C═N—, —N═C— and phenylene; and/or optionally substituted by at least one atom or radical selected from the group consisting of halogen, —OR₅, —SR₅, epoxy, epithio, oxo and —COOR₅, wherein R₅ is H or (C1-C4) alkyl.

9. A conjugate according to claim 1, wherein X₁ is phenylene optionally substituted by one or more halogen, (C₁-C₄) alkyl, —OR₅, —SR₅, or —COOR₅ groups, wherein R₅ is H or (C₁-C₄) alkyl.

10. A conjugate according to claim 8, wherein X₁ is an unsaturated C₂-C₁₀ aliphatic chain containing one or more double and/or one or more triple bonds.

11. A conjugate according to claim 9, wherein X₁ is —C≡C— or phenylene and X₂ is a covalent bond.

12. A conjugate according to claim 9, wherein X₁ is —C≡C— or phenylene and X₂ is phenylene.

13. A conjugate according to claim 1 of formula I selected from the group consisting of formulas Ia to 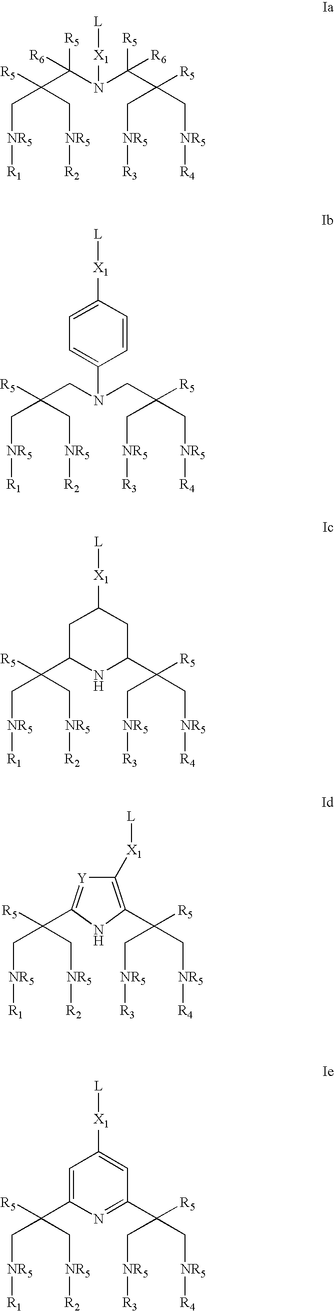
wherein
L is a moiety of a ligand of a receptor associated with malignant tumors or a chemotherapeutic drug moiety;
X₁ is a C₂-C₁₀ hydrocarbylene chain;
R₁ to R₄ each is selected from the group consisting of H, (C₁-C₄) alkyl and —CH₂R₇;
R₅ and R₆ each is H or (C₁-C₄) alkyl;
R₇ is selected from the group consisting of —COOR₈, —COO⁻, 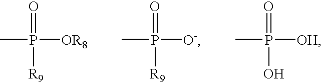
—PO₃²⁻ and —CONHR₈;
R₈ and R₉ each is selected from the group consisting of H, (C₁-C₄) alkyl, phenyl and benzyl, wherein the phenyl or benzyl can be substituted by at least one group selected from the group consisting of halogen, (C1-C4) alkyl and OR₅;
Y is selected from the group consisting of C, N, and S;
and complexes of the conjugates of formula Ia to Ie,
wherein R₁ to R₄ each is —CH₂R₇ and R₇ is —COOR₈, —COO⁻, PO₂(R)(R₉), PO₃²⁻, PO₃H₂ or —P(R₉)O₂⁻ with a paramagnetic lanthanide metal selected from the group consisting of Gd (III), Eu(III), Dy(III), Tb(III), Tm(III), Yb(III) and Pr(III), and of a conjugate of formula I, II or III, wherein R, to R₄ each is H, (C₁-C₄) alkyl or —CH₂R₇ and R₇ is as defined above, with a paramagnetic transition metal selected from the group consisting of Mn(II), Co(III), Ni(III), Fe(II) and Fe(III).

14. A conjugate according to claim 1 of formula II selected from the group consisting of formulas IIa to IIe: 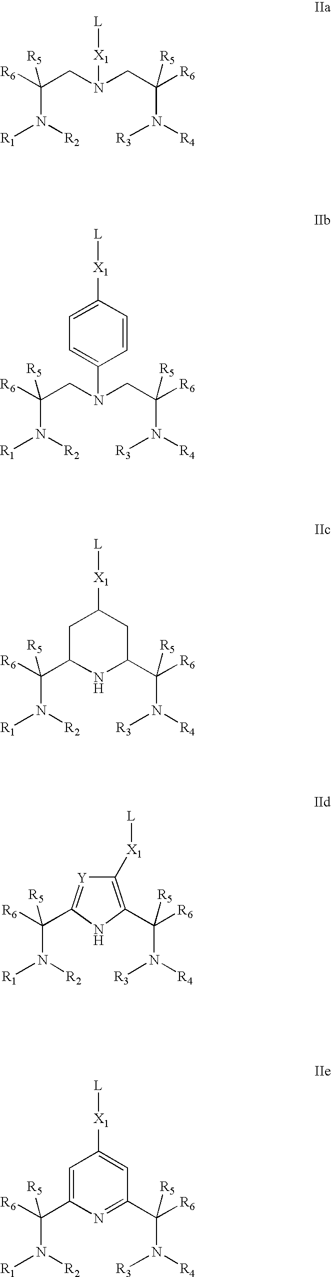
wherein
L is a moiety of a ligand of a receptor associated with malignant tumors or a chemotherapeutic drug;
is a C₂-C₁₀ hydrocarbylene chain;
R₁ to R₄ each is selected from the group consisting of H, (C₁-C₄) alkyl and —CH₂R₇;
R₅ and R₆ each is H or (C₁-C₄) alkyl;
R₇ is selected from the group consisting of —COOR₈, —COO⁻, 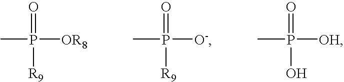
—PO₃²⁻ and —CONHR₈;
R₈ and R₉ each is selected from the group consisting of H, (C₁-C₄) alkyl, phenyl and benzyl, wherein the phenyl or benzyl can be substituted by at least one group selected from the group consisting of halogen, (C₁-C₄) alkyl and OR₅;
Y is selected from the group consisting of C, N, and S; and complexes of the conjugates of formula IIa to IIe, wherein R₁ to R₄ each is —CH₂R₇ and R₇ is —COOR₈, —COO⁻, PO₂(R₈)(R₉), PO₃²⁻, PO₃H₂ or —P(R)O₂⁻ with a paramagnetic lanthanide metal selected from the group consisting of Gd (III), Eu(III), Dy(III), Tb(III), Tm(III), Yb(III) and Pr(III), and of a conjugate of formula I, II or III, wherein R₁ to R₄ each is H, (C₁-C₄) alkyl or —CH₂R₇ and R₇ is as defined above, with a paramagnetic transition metal selected from the group consisting of Mn(II), Co(III), Ni(III), Fe(II) and Fe(III).

15. A conjugate according to claim 1 of formula III selected from the group consisting of formulas IIIa to IIIf: 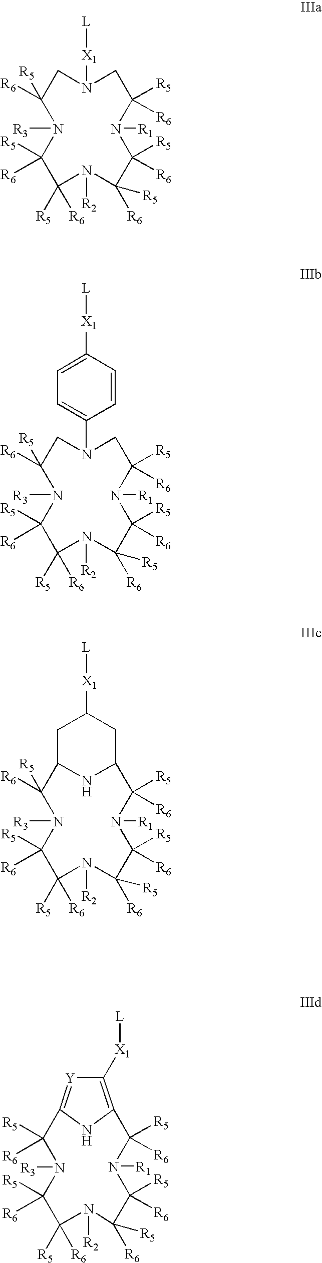 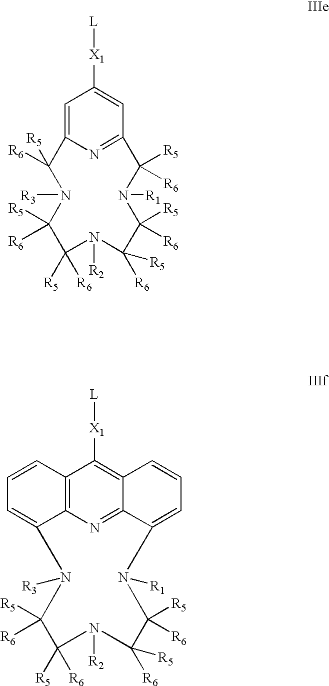
wherein
L is a moiety of a ligand of a receptor associated with malignant tumors or a chemotherapeutic drug moiety;
X₁ is a C₂-C₁₀ hydrocarbylene chain;
R₁ to R₃ each is selected from the group consisting of H, (C₁-C₄) alkyl and —CH₂R₇;
R₅ and R₆ each is H or (C₁-C₄) alkyl;
R₇ is selected from the group consisting of —COOR₈, —COO⁻ 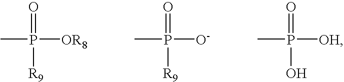
—PO₃²⁻ and —CONHR₈;
R₈ and R₉ each is selected from the group consisting of H, (C₁-C₄) alkyl, phenyl and benzyl, wherein the phenyl or benzyl can be substituted by at least one group selected from the group consisting of halogen, (C1-C4) alkyl and OR₅;
Y is selected from the group consisting of C, N, and S;
and complexes of the conjugates of formulas IIIa to IIIf, wherein R₁ to R₃ each is —CH₂R₇ and R₇ is —COOR₈, —COO⁻, PO₂(R₈)(R₉), PO₃²⁻, PO₃H₂ or —P(R₉)O₂⁻ with a paramagnetic lanthanide metal selected from the group consisting of Gd (III), Eu(III), Dy(III), Tb(III), Tm(III), Yb(III) and Pr(III), and of a conjugate of formula I, II or III, wherein R, to R₄ each is H, (C₁-C₄) alkyl or —CH₂R₇ and R₇ is as defined above, with a paramagnetic transition metal selected from the group consisting of Mn(II), Co(III), Ni(III), Fe(II) and Fe(III).

16. A conjugate according to claim 13, wherein L is a moiety of a ligand of a steroid receptor associated with malignant tumors.

17. A conjugate according to claim 16, wherein said steroid receptor is an estrogen receptor and said ligand is selected from the group consisting of 17β-estradiol, estrone, estriol, tamoxifen and analogs of the foregoing.

18. A conjugate according to claim 16, wherein said steroid receptor is the progesterone receptor and said ligand is a progestin such as progesterone.

19. A conjugate according to claim 16, wherein said steroid receptor is the androgen receptor and said ligand is testosterone.

20. A conjugate according to claim 13, wherein L is a moiety of a ligand of a non-steroidal hormone receptor associated with malignant tumors or another abnormalities such as luteinizing hormone/human chorionic gonadotropin receptors, human growth hormone receptor, and somatostatin receptor, or L is a ligand of another receptor such as a retinoic acid receptor.

21. A conjugate according to claim 13, wherein L is a moiety of a chemotherapeutic drug.

22. A conjugate according to claim 13, wherein X₁ is a saturated or unsaturated aliphatic C₂-C₁₀ hydrocarbylene chain optionally interrupted by at least one atom or radical selected from the group consisting of —O—, —S—, —N(R₅)—, —CO—N(R₅)—, —N(R₅)—CO—, —COO—, —OOC—, —N═N—, —C═N—, —N═C— and phenylene; and/or optionally substituted by at least one atom or radical selected from the group consisting of halogen, —OR₅, —SR₅, epoxy, epithio, oxo and —COOR₅, wherein R₅ is H or (C1-C4) alkyl.

23. A conjugate according to claim 22, wherein X₁ is an unsaturated C₂-C₁₀ aliphatic chain containing one or more double and/or one or more triple bonds, preferably —C≡C—.

24. A conjugate according to claim 13, wherein X₁ is phenylene optionally substituted by one or more halogen, (C₁-C₄) alkyl, —OR₅, —SR₅, or —COOR₅ groups, wherein R₅ is H or (C₁-C₄) alkyl.

25. A conjugate according to claim 13 of formula Ia to Ie, wherein L is a 17β-estradiol or tamoxifen moiety, and X₁ is —C≡C—.

26. The conjugate according to claim 25 of formula Ie, wherein L is a 17β-estradiol or tamoxifen moiety X₁ is —C≡C—, R₁ to R₃ each is —CH₂R₇, wherein R₇ is selected from the group consisting of —COOR₈, —COO⁻, PO₂(R₈)(R₉), PO₃²⁻, PO₃H₂ and —P(R₉)O₂⁻, R₄, R₅ and R₆ each is H, and R₈ and R₉ each is H or (C₁-C₄) alkyl, and a complex thereof with a paramagnetic lanthanide metal selected from the group consisting of Gd (III), Eu(III), Dy(III), Tb(III), Tm(III), Yb(III) and Pr(III), preferably Gd(III).

27. The conjugate according to claim 25 of formula Ie, wherein L is 17β-estradiol or tamoxifen, X₁ is —C≡C—, R₁ to R₄ each is —CH₂COOR₈ or —CH₂COO⁻, R₅ and R₆ each is H and R₈ is H or (C₁-C₄) alkyl, and a complex thereof with a paramagnetic lanthanide metal selected from the group consisting of Gd (III), Eu(III), Dy(III), Tb(III), Tm(III), Yb(III) and Pr(III, preferably Gd(III) or Eu (III).

28. The conjugate according to claim 25 of formula Ie, wherein L is a 17β-estradiol or tamoxifen moiety, X₁ is —C≡C—, and R₁ to R₆ each is H, and a complex thereof with a paramagnetic transition metal selected from the group consisting of Mn(II), Co(III), Ni(III), Fe(II) and Fe(III), preferably Fe(III).

29. A conjugate according to claim 14 of formula IIa to IIe, wherein L is a 17β-estradiol or tamoxifen moiety, and X₁ is —C≡C—.

30. The conjugate according to claim 29 of formula IIe, wherein R₁ to R₄ each is —CH₂R₇, wherein R₇ is selected from the group consisting of —COOR₈, —COO⁻, PO₂(R₈)(R₉), PO₃²⁻, PO₃H₂ and —P(R₉)O₂⁻, R₅ and R₆ each is H, and R₈ is H or (C₁-C₄) alkyl and a complex thereof with a paramagnetic lanthanide metal selected from the group consisting of Gd (III), Eu(III), Dy(III), Tb(III), Tm(III), Yb(III) and Pr(III).

31. The conjugate according to claim 30 of formula IIe, wherein L is a 17β-estradiol moiety, R₅ and R₆ each is H, and R₁ to R₄ each either is —CH₂COO-tBut (compound 5) or —CH₂COOH (compound 6).

32. The conjugate according to claim 30 of formula IIe, wherein L is a tamoxifen moiety, R₅ and R₆ each is H, and R₁ to R₄ each either is —CH₂COO-tBut (compound 14) or —CH₂COOH (compound 15).

33. The conjugate complex according to claim 30 of the conjugate of formula IIe, wherein L is a 17β-estradiol or tamoxifen moiety, R₁ to R₄ each is —CH₂COO⁻, and R₅ and R₆ each is H, with a paramagnetic lanthanide metal selected from the group consisting of Gd (III), Eu(III), Dy(III), Tb(III), Tm(III), Yb(III) and Pr(III).

34. The conjugate complex of claim 33, wherein said paramagnetic lanthanide metal is Gd(III) and L is 17β-estradiol (compound 7) or tamoxifen (compound 16).

35. A conjugate according to claim 15 of formula IIIa to IIIf, wherein L a is 17β-estradiol or tamoxifen moiety, X₁ is —C≡C—, R₁ to R₃ each is —CH₂R₇, wherein R₇ is selected from the group consisting of —COOR₈, —COO⁻, PO₂(R₈)(R₉), PO₃²⁻, PO₃H₂ and —P(R)O₂⁻, R₅ and R₆ each is H, and R₈ is H or (C₁-C₄) alkyl, preferably H, and a complex thereof with a paramagnetic lanthanide-metal selected from the group consisting of Gd(III), Eu(III), Dy(III), Tb(III), Tm(III), Yb(III) and Pr(III), preferably Gd(III) or Eu(III).

36. A method of using a contrast agent to obtain magnetic resonance images of a patient, comprising: administering to a patient a magnetic resonance imaging contrast agent comprising a paramagnetic metal complex of a conjugate of the formula I, II or III as defined in claim 1, and taking magnetic resonance images of the patient prior to said administration and thereafter.

37. A molecular magnetic resonance imaging (MRI) method comprising the steps of: (i) administering to a patient a MRI contrast agent comprising a paramagnetic metal complex of a conjugate of formula I, II or III as defined in claim 1; and (ii) subjecting the patient to magnetic resonance imaging by generating at least one MR image of the target region of interest within the patient's body prior to said administration and one or more MR images thereafter.

38. The MRI method according to claim 37 for tumor diagnosis comprising the steps of: (i) administering to a patient suspected of having a tumor said MRI contrast agent, wherein L is a moiety of a ligand of a receptor associated with said tumor; (ii) generating an MR image at zero time and at a second or more time points thereafter; and (iii) processing and analyzing the data to diagnose the presence or absence of a tumor.

39. The MRI method according to claim 37 for prognosticating the effectiveness of a chemotherapeutic or hormonal treatment of a patient bearing a malignant tumor and being treated with a chemotherapeutic or hormonal agent, comprising the steps of: (i) administering to the patient said MRI contrast agent, wherein L is a moiety of a ligand of a receptor associated with the tumor; (ii) generating an MR image at zero time and at a second or more time points thereafter; and (iii) processing and analyzing the data to prognosticate the effectiveness of the chemotherapeutic or hormonal agent in the treatment of said tumor in said patient.

40. The MRI method according to claim 37 for follow-up of malignant tumor therapy in a patient by a chemotherapeutic or hormonal agent, comprising the steps of: (i) administering to the patient said MRI contrast agent, wherein L is a moiety of a ligand of a receptor associated with the tumor; (ii) generating an MR image at zero time and at a second or more time points thereafter; and (iii) processing and analyzing the data to evaluate the effectiveness of the chemotherapeutic or hormonal agent in the treatment of said tumor in said patient.

41. The MRI method according to claim 37 for monitoring a chemotherapeutic drug or an anti-hormonal agent delivery to a malignant tumor, comprising the steps of: (i) administering to the patient said MRI contrast agent, wherein L is a moiety of a ligand of a receptor associated with the tumor; (ii) generating an MR image at zero time and at a second or more time points thereafter; and (iii) processing and analyzing the data to evaluate the effectiveness of the delivery of the chemotherapeutic or anti-hormonal agent to said tumor.

42. A method according to claim 36 wherein said tumor is breast or prostate cancer.

43. A method according to claim 36 wherein the MRI contrast agent is the Gd(III) complex herein designated compound 7 or the Gd(III) complex herein designated compound 16.

44. The method according to claim 37 wherein said MRI contrast agent is administered by a bolus intravenous injection.

45. The method according to claim 37 wherein said MRI contrast agent is administered by slow infusion.

46. An MRI contrast agent comprising a paramagnetic metal complex of a conjugate of formula I, II or III as defined in claim 1.

47. The MRI contrast agent according to claim 46 wherein said metal complex is the Gd(III) complex compound 7 or compound 16.

48-52. (canceled)
